(19) United States
(12) Patent Application Publication
(10) Pub. No.: US 2022/0127580 A1
(43) Pub. Date: Apr. 28, 2022

(54) COMPOSITIONS AND METHODS FOR REPROGRAMING NON-HEPATOCYTE CELLS INTO HEPATOCYTE CELLS

(71) Applicant: Peking University, Beijing (CN)

(72) Inventors: Hongkui Deng, Beijing (CN); Bingqing Xie, Beijing (CN); Da Sun, Beijing (CN); Yuanyuan Du, Beijing (CN)

(21) Appl. No.: 17/434,289

(22) PCT Filed: Feb. 26, 2019
(86) PCT No.: PCT/CN2019/076180

§ 371 Date: Aug. 26, 2021

(51) Int. Cl. C12N 5/071; C12N 15/113

(52) U.S. Cl. CPC C12N 5/0672; C12N 15/1135; C12N 2310/14; C12N 2501/01; C12N 2510/00; C12N 2501/65; C12N 2501/727; C12N 2502/45; C12N 2506/02; C12N 2501/15

(57) ABSTRACT

A method for inducing non-hepatocytes into hepatocyte-like cells, wherein the non-hepatocytes are induced to express or overexpress hepatic fate conversion and maturation factors, cultured in somatic cell culture medium, hepatocyte expansion culture medium and 2C medium for a sufficient period of time to convert the non-hepatocyte cell into cells with hepatocyte-like properties, are provided. The iHeps induced according to the methods are also provided.

### FIELD OF THE INVENTION

The present invention generally relates to use of transcription factors as well as small molecules for reprograming eukaryotic cells into cells with hepatocyte-like characteristics.

### BACKGROUND OF THE INVENTION

Recent advances in reprogramming using various transcription factors have allowed the conversion between mature somatic cell types.

Previous studies identified a combination of HNF1A, HNF4A, HNF6, ATFS, PROX1, and CEBPA for obtaining hepatocyte-like cells from a non-hepatocyte cell using a defined cell culture scheme. Du, et al., Cell Stem Cell, 14:394-403 (2014).

However, current approaches attempting to convert one cell lineage directly to another suffer from several shortcomings, including the residual memory of the initial cells and limited functional conversion of target cells (Cahan, et al., Cell, 158:903-915 (2014)). Zaret, et al., noted the epigenetic barriers to direct reprogramming such as the packed H3K9me3 heterochromatin domains, which are hard for transcription factors (TFs) to access for activating tissue-specific genes of the target cells, resulting in incomplete cell fate conversion (Becker, et al., Trends in Genetics, 32:29-41 (2016)). Other studies observed only limited activation of key hepatic genes located in the H3K9me3 domain by direct fibroblast-to-hepatocyte conversion strategy (Gao, et al., Stem Cell Reports, 9:1813-1824 (2017); Becker, et al., Mol. Cell., 68:1023-1037 e1015 (2017)).

There is therefore a need for a method inducing non-hepatocyte cells into functional induced hepatocytes that show improved hepatocyte functional activity, when compared to known induced hepatocytes.

It is therefore an object of the present invention to provide a method of inducing conversion of a non-hepatocyte cell, into an induced hepatocyte cell (iHep) with metabolic function.

It is also an object of the present invention to provide induced hepatic cells with metabolic function.

It is still an object of the present invention to provide a method using induced hepatocytes for drug development, bioartificial liver system and in vivo and in-vitro hepatic applications.

It is further an object of the present invention to provide kits for reprograming a non-hepatocyte into an iHep.

### SUMMARY OF THE INVENTION

A method for inducing reprograming of a cell of a first type, which is not a hepatocyte (i.e., non-hepatocyte cells), into a hepatocyte-like cell, as indicated by functional hepatic drug metabolizing and transporting capabilities, is disclosed. These cells are denoted herein as induced hepatocytes (iHeps). The non-hepatocyte is treated to upregulate hepatocyte inducing factors, cultured in somatic cell culture medium (transformation phase), expanded in hepatocyte cell culture medium (expansion phase) and further cultured in 2C medium (maturation phase) for a sufficient period of time to convert the cell into cells with hepatocyte-like properties.

The reprograming method includes two phases, a hepatic progenitor cell generation phase (Phase I) and an induced hepatocyte (iHep) generation phase (Phase II). Phase I includes the steps of: (a) treating the cells to upregulate hepatocyte inducing factors and MYC, and downregulate p53 and culture the cells in cell culture medium (transformation phase) and (b) replating and culturing the cells in HEM (Hepatic Expansion Medium) (expansion phase). Phase II includes culturing the cells in a custom differentiation medium for example, the 2C medium disclosed herein. Induced hepatocytes (iHeps) are obtained following this cell culture scheme.

In Phase I(a), the non-hepatocyte cell is preferably transformed to overexpress the following hepatocyte inducing factors: Hematopoietically-expressed homeobox protein (HHEX), Hepatocyte nuclear factor 4-alpha (HNF4A), Hepatocyte nuclear factor 6-alpha A (HNF6A), GATA4 and forkhead box protein A2 (FOXA2), MYC, and downregulate p53 gene expression and/or protein activity. Non-hepatocytes (treated to upregulate hepatocyte inducing factors, and MYC and to downregulate p53) are then cultured and expanded in vitro in HEM (Hepatic Expansion Medium) (expansion phase) and hepatic progenitor cells were generated in Phase 1. These hepatic progenitor cells further matured in a cell culture medium supplemented with at least one cyclic AMP agonist and at least one TGFβ receptor inhibitor (termed as 2C), to obtain iHeps in Phase II (maturation phase).

The cells are identified as iHeps, based on known structural and functional properties of hepatocytes.

Also disclosed are functional induced hepatocytes (iHeps). In a preferred embodiment, the induced hepatocytes are human induced hepatocytes (hiHeps). iHeps express at least one hepatocyte marker selected from the group consisting of albumin, Cytochrome P450 (CYP)3A4, CYP2B6, CYP2C8, CYP2C9, CYP2C19, CYP2D6, CYP1A2, CYP2A6, UGT1A1 and POR. In a preferred embodiment, iHeps express all ten markers, CYP3A4, CYP2B6, CYP2C8, CYP2C9, CYP2C19, CYP2D6, CYP1A2, CYP2A6, UDP glucuronosyltransferase (UGT)1A1 and POR.

Kits for inducing reprograming of non-hepatocytes cells into iHeps are also disclosed. The kit includes factors which upregulate the hepatocyte inducing factors disclosed herein, and MYC and downregulate p53 gene expression and/or protein levels.

### BRIEF DESCRIPTION OF THE DRAWINGS

FIGS. 1A-H show generation of human hepatic progenitor cell-like cells (hHPLCs) from fibroblasts by defined factors. FIG. 1A shows a scheme of the two-step reprogramming process. FIG. 1B shows quantification of reprogrammed ALB+ cells in different hepatic progenitor cell (HPC) culture media at 15 dpi. n=2. ** P<0.01. FIG. 1C shows quantification of reprogrammed ALB+ cells in HEM at different time points. n=3. FIG. 1D shows flow cytometry analysis of ALB positive cells in hHPLCs at P7 and P27.

FIG. 1E shows RT-qPCR analysis of hHPLCs, human embryonic fibroblasts (HEFs) and human fetal liver cells (hFLCs) for hHPC markers. n=2. FIG. 1F shows dynamic gene expression analysis of the hHPC markers ALB, AFP, EPCAM as well as the fibroblast markers COL1A1 and THY1 in reprogrammed cells at different time points by RT-qPCR. n=2. FIG. 1G shows hierarchical clustering of global gene expression of HEFs, hFLCs, F-PHHs and hHPLCs at different passages. FIG. 1H shows population doubling time of hHPLCs at P5 and P30 as well as of HEFs at P3 and P10. n=3. Scale bars=50 μm. Data are presented as mean±SEM.

FIG. 2A shows RT-qPCR analyses of key hepatic functional markers and transcription factors in 2C-cultured hepatocytes (n=3), adult liver tissues (ALs, n=3) and hepG2 cells (n=2). The gene expression level was normalized to ALs (top panel); The bottom panel shows drug-metabolizing activity analyses of two batches of cultured hepatocytes at 30 days. Mass spectrometry was used to analyze the activities of CYP3A4, CYP1A2, CYP2C9 and CYP2D6. The following CYP450-specific substrates were used: CYP3A4-T (testosterone), CYP1A2 (phenacetin), CYP2C9 (diclofenac) and CYP2D6 (dextromethorphan). n=3. The scale bars represent 50 μm. Data are presented as mean±SEM. Data not detected, “n.d.”. FIG. 2B shows RT-qPCR analysis of hepatic transcription factors in HEFs (n=3), HepG2 cells (n=2), hiHeps (n=2) and F-PHHs (n=5). Relative expression was normalized to HEFs. FIG. 2C shows flow cytometry analysis of ALB+ cells in hiHeps. FIG. 2D shows RT-qPCR analysis of major mature hepatocyte functional genes in HepG2 cells (n=2), hHPLCs (n=2), hiHeps (n=8), and F-PHHs (n=5) and adult liver tissues (ALs, n=4). Relative expression was normalized to F-PHHs. FIG. 2E shows dynamic expression of key hepatic genes in hiHeps for every 5 days up to 40 days cultured in 2C medium by RT-qPCR. n=3. FIG. 2F is a line graph showing ELISA analysis of AFP secretion in hiHeps for every 5 days up to 40 days. n=3. FIG. 2G shows albumin secretion in HEFs, hiHeps and PHHs by ELISA. n=3. FIG. 2H shows dynamic monitoring of albumin secretion in hiHeps and PHHs. FIG. 2I shows hierarchical clustering of global gene expression of HepG2 cells, HEFs, F-PHHs, AL and hiHeps derived from hHPLCs at different passages. Asterisk represents PHHs and AL from the same donors.

FIGS. 3A-D show comparable CYP drug-metabolizing activities and toxicity prediction ability of hiHeps to PHHs. FIG. 3A is a bar graph showing mass spectrometry analysis for the drug-metabolic activities of seven CYP450s in hiHeps, HepG2 cells and F-PHHs. n=3. FIG. 3B is a bar graph showing induction activities of CYP450s in response to rifampin, f3-naphthoflavone, lansoprazole or phenobarbital. n=3. FIG. 3C is shows comparison of the TC50 values in hiHeps, PHHs and HepG2 cells with 25 compounds. (Gray region: less than 2.5-fold difference. n=3 for all compounds in hiHeps, PHHs and HepG2 cells, except that n=2 for Aflatoxin B1 (AFB1) in PHHs. r: Pearson correlation coefficient.) FIG. 3D is a line graph showing time- and dose-dependent chronic toxicity of troglitazone in hiHeps. n=6. FIG. 3E shows hierarchical clustering of global CpG methylation pattern of hiHeps, F-PHHs and HEFs. “n” represent the number of the CpG sites and hierarchical clustering of differentially methylated CpG sites in hiHeps, F-PHHs and HEFs. FIG. 3F shows RT-qPCR of key hepatic microRNAs in HEFs, HepG2 cells, hiHeps, and F-PHHs. FIG. 3G shows hierarchical clustering of global gene expression of HEFs, HepG2 cells, hiHeps derived from fibroblasts of different donors, F-PHHs and AL. The asterisk represents F-PHHs and AL from the same donor. FIG. 3H shows the principle component analysis of global gene expression of HEFs, HepG2 cells, hiHeps derived from fibroblasts of different donors, F-PHHs and AL. The asterisk represents F-PHHs and AL from the same donor. FIG. 3I shows RT-qPCR analysis of major mature hepatocyte functional genes in HEFs (n=3), hiHeps (n=3) derived from CRL-2097 and F-PHHs (n=5). Relative expression was normalized to F-PHHs.

FIG. 4A is a bar graph showing UPLC/MS/MS analysis of drug-metabolic activities of 7 CYP450s in hiHeps derived from CRL-2097, HepG2 cells and F-PHHs. Results are presented as pmol/min per million cells. n=3. FIG. 4B is a bar graph showing induction of CYP3A4 (testosterone), CYP1A2 (phenacetin) and CYP2B6 (bupropion) activities in response to rifampin, β-naphthoflavone, lansoprazole and phenobarbital in hiHeps derived from CRL-2097, HepG2 cells and PHHs by UPLC/MS/MS. The scale bars represent 50 μm. Data are presented as mean±SEM. FIG. 4C shows fold changes of CYP3A4 expression in hiHeps in response to structurally different inducers. Expression was normalized to vehicle-treated controls. Data are presented as mean±SEM. FIG. 4D show dose-dependent viability curves of hiHeps, F-PHHs and HepG2 cells treated with AFB1. The concentration that was calculated to cause a 50% decrease in cell viability (brown line) was determined as the TC50. All data were normalized to cultures treated with vehicle control. FIGS. 4E and 4F show quantification of dose-dependent steatosis and phospholipidosis in hiHeps after exposure to steatosis/phospholipidosis-causing compounds (FIG. 3E), rifampin (non-steatosis/phospholipidosis-causing compound) or DMSO. n=4; a.u., arbitrary units. FIG. 4G shows drug-drug interactions mediated toxicity. Toxicity of AFB1 (presented by TC50 value) and flutamide (presented by cell viability at 0.3 mM or 3 mM) in hiHeps, and HepG2 cells after treatment with DMSO, the CYP3A4 inducer rifampin (RIF) or the combination of RIF and CYP3A4 inhibitor ketoconazole (KC). n=3 for AFB1 and n=6 for flutamide in both cell types. Data are presented as mean±SEM. One-way ANOVA was performed. *P<0.05; ** P<0.01; *** P<0.001. In these figure, top panels (left and right) showed the concentration which would lead to 50% cells death of hiHeps or HepG2, and the column represented concentration. Bottom panel (left and right) showed the cell viability for hiHeps at 0.3 mM of flumaide and the cell viability for HepG2 at 3 mM of flumaide. As at 0.3 mM of flumaide, HepG2 were 100% alive, so we showed the cell viability of HepG2 at 3 mM which could lead to the death of HepG2.

FIG. 5A shows dynamic gene expression analysis of NTCP in hiHeps for 35 days by RT-qPCR. n=3. FIG. 5B shows quantification of HBV markers in hHPLCs, hiHeps, PHHs and HepG2-NTCP cells in 7 days post infection and uninfected hiHeps. n=3. FIG. 5C shows a southern blot analysis of cccDNA in hiHeps. FIGS. 5D-G show dynamics expression of different HBV markers. HBV proteins FIG. 5D), HBV-RNA (FIG. 5E), supernatant HBV-DNA (FIG. 5F) and intracellular HBV-DNA (FIG. 5G) were analyzed in HBV-infected hiHeps and hiHeps treated with ETV, LAM and IFN-α. n=3. FIG. 5H shows gene expression analysis of key ISGs in HBV-infected hiHeps, hiHeps treated with IFN-α, uninfected hiHeps and those treated with IFN-α. n=3. Scale bars=50 μm. Data are presented as mean±SEM. FIG. 5I shows dynamics expression of different HBV markers in 30 days post infection. HBV proteins, HBV-RNA, supernatant HBV-DNA and intracellular HBV-DNA were analyzed in HBV-infected hiHeps and hiHeps treated with a viral entry inhibitor N-terminal myristoylated peptides (MYR). n=3. The scale bars represent 50 μm. Data are presented as mean±SEM.

FIG. 6A shows gene expression analysis of key hHPC markers in hHPLCs before and after cryopreservation (left) as well as key hepatocyte functional markers in hiHeps derived from pre-cryopreserved and post-cryopreserved hHPLCs (right) by RT-qPCR. n=3. Data are presented as mean±SEM. FIG. 6B contains violin plots showing the activation levels of 261 hepatic genes that were silent and marked by H3K9me3 in fibroblasts. RNA-seq data (GSE103078) from a previous direct fibroblast-to-hepatocyte lineage reprogramming study (left) and this study with a novel two-step lineage reprogramming strategy (right) were analyzed. The RNA levels in hiHeps from two different strategies were plotted on a relative scale ranging from the fibroblast levels (0%) to primary human hepatocyte levels (100%) using log 2-transformed values. That the relative genes expression levels are higher than 50% was regarded as activated. n=2.

### DETAILED DESCRIPTION OF THE INVENTION

### I. Definitions

“2C medium” as use herein refers to basal cell culture medium for hepatocytes supplemented with one or more cAMP signaling activators and one or more TGFβ receptor inhibitor, for example, HCM (hepatocyte culture medium) or William's E medium containing 2% B27, 1% GlutaMAX, supplemented with forskolin and SB431542.

As used herein a “culture” means a population of cells grown in a medium and optionally passaged. A cell culture may be a primary culture (e.g., a culture that has not been passaged) or may be a secondary or subsequent culture (e.g., a population of cells which have been subcultured or passaged one or more times).

As used herein, “downregulation” or “downregulate” refers to the process by which a cell decreases the quantity and/or activity of a cellular component, for example, DNA, RNA or protein, in response to an external variable.

As used herein, “functional induced hepatocytes (iHeps)” refers to induced hepatocytes which show the expression of at least one of CYP3A4, CYP2B6, CYP2C8, CYP2C9, CYP2C19, CYP2D6, CYP1A2, CYP2A6, UGT1A1 or POR, at levels comparable to the expression of the same enzyme in freshly isolated primary human hepatocytes (F-PHHs) obtained from liver.

As used herein, the term “host cell” refers to non-hepatocytes eukaryotic cells into which a recombinant nucleotide, such as a vector, can be introduced.

The term “induced hepatocytes” (iHeps) as used herein refers to cells which are not naturally occurring hepatocytes, and which are artificially derived from non-hepatocyte cells.

The terms “oligonucleotide” and “polynucleotide” generally refer to any polyribonucleotide or polydeoxribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C

As used herein, “transformed” and “transfected” encompass the introduction of a nucleic acid (e.g. a vector) into a cell by a number of techniques known in the art.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

“Reprogramming” as used herein refers to the conversion of a one specific cell type to another. For example, a cell that is not a hepatocyte cab be reprogrammed into a cell that is morphologically and functionally like a hepatocyte.

As used herein “treating a cell/cells” refers to contacting the cell(s) with factors such as the nucleic acids disclosed herein to downregulate or upregulate the quantity and/or activity of a cellular component, for example, DNA, RNA or protein. This phrase also encompasses contacting the cell(s) with any factors including proteins and small molecules that can downregulate or upregulate the gene/protein of interest.

The term “upregulate expression of” means to affect expression of, for example to induce expression or activity, or induce increased/greater expression or activity relative to an untreated cell.

As used herein, “upregulation” or “upregulate” refers to the process by which a cell increases the quantity and/or activity of a cellular component, for example, DNA, RNA or protein, in response to an external variable.

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

### II. Compositions

A. Factors Inducing Non-Hepatocyte Cells into Cells with Hepatocyte Properties

Obtaining fully functional hepatocytes from non-hepatocyte sells remains a challenge, especially from differentiated cells. Functional human induced hepatocytes (hiHeps) can be generated from fibroblasts by upregulating mRNA (or levels of protein encoded by the mRNA) levels of the following factors: Hematopoietically-expressed homeobox protein, HHEX; hepatocyte nuclear factors, HNF4A, HNF6A; GATA-binding protein, GATA4 and forkhead box protein, FOXA2, as well as MYC genes, and downregulating p53 expression of levels in a non-hepatocyte cell, followed by a defined cell culture protocol disclosed herein. All known functional variants and isoforms of the hepatocyte inducing factors disclosed herein are contemplated.

These known sequences are readily available from the National Center for Biotechnology Information Genebank database. Genebank accession numbers for HHEX, HNF4A, HNF6A, GATA4 and FOXA2 are listed in Table 1.

In some embodiments, the method includes selecting FOXA1 or FOXA3 as the forkhead box protein genes/protein to be upregulated, in place of FOXA2 or in combination with FOXA2. Some preferred embodiments do not include upregulating expression of FOXA1 or FOXA3, when FOXA2 is upregulated.

In some embodiments the method includes selecting GATA6 as the GATA-binding protein gene/protein to be upregulated, in place of GATA 4, or in combination with GATA4. Some preferred embodiments do not include upregulating expression of GATA6 when GATA4 is upregulated.

Preferably, p53 activity is additionally, downregulated as indicated by a downregulation of the p53 gene, mRNA and/or protein levels.

i. HHEX

The HHEX gene encodes Hematopoietically-expressed homeobox protein HHEX.

The HHEX transcription factor acts as a promoter in some instances and an inhibitor others. It interacts with a number of other signaling molecules to play an important role in the development of multiple organs, such as the liver, thyroid and forebrain. The importance of this transcription factor is illustrated by the inability of HHEX knockout mice embryos to survive gestation.

An exemplary HHEX gene is represented by NM_002729.4:

ggataaatgt agcgccgcgg cgcgggccag
cagctctgcg aggggccgga
gcgcggcgga gccatgcagt acccgcaccc
cgggccggcg gcgggcgccg
tgggggtgcc gctgtacgcg cccacgccgc
tgctgcaacc cgcacacccg
acgccctttt acatcgagga catcctgggc
cgcgggcccg ccgcgcccac
gcccgccccc acgctgccgt cccccaactc
ctccttcacc agcctcgtgt
ccccctaccg gaccccggtg tacgagccca
cgccgatcca tccagccttc
tcgcaccact ccgccgccgc gctggccgct
gcctacggac ccggcggctt
cgggggccct ctgtacccct tcccgcggac
ggtgaacgac tacacgcacg
ccctgctccg ccacgacccc ctgggcaaac
ctctactctg gagccccttc
ttgcagaggc ctctgcataa aaggaaaggc
ggccaggtga gattctccaa
cgaccagacc atcgagctgg agaagaaatt
cgagacgcag aaatatctct
ctccgcccga gaggaagcgt ctggccaaga
tgctgcagct cagcgagaga
caggtcaaaa cctggtttca gaatcgacgc
gctaaatgga ggagactaaa
acaggagaac cctcaaagca ataaaaaaga
agaactggaa agtttggaca
gttcctgtga tcagaggcaa gatttgccca
gtgaacagaa taaaggtgct
tctttggata gctctcaatg ttcgccctcc
cctgcctccc aggaagacct
tgaatcagag atttcagagg attctgatca
ggaagtggac attgagggcg
ataaaagcta ttttaatgct ggatgatgac
cactggcatt ggcatgttca
gaaaactgga tttaggaata atgttttgct
acagaaaatc ttcatagaag
aactggaagg ctatataaga aagggaatca
attctctggt attctggaaa
cctaaaaata tttggtgcac tgctcaatta
acaaacctac atggagacct
taattttgac ttaacaaata gtttatgtac
tgctcttagg ttgttttgat
aaagtgacat tatagtgatt aaattcttcc
ccctttaaaa aaacagttag
tggttttcac tatttataaa aaattaattt
tgaacttttt gttaaatttt
taagttatag ctttaaaggt tttaatagga
ccttcttgaa cgacttttct
gtaatctgtt tatctcccac ttaatggaaa
ggcaaagggg taccccaaat
ccagaggtgc ctacatttca ggcagccttg
gagtatttta aaaggaaaac
attctttact tttatatgac attcttatac
tgctgtctca aatccaaaaa
catttcagag ctcttgtctc agagatgtgt
gttctttttg tcagagatat
ggttgatgag aatcttaaat gcttgttttg
cactatcact tagtacctgt
ttgaccaagg tgttaagggg atagtacctc
ccaattcaag cagagaaact
gacctgacta aagttaatcg cagatgaact
agaagtcaca ggttaattaa
atgtaagtag attgtagata ctgttttata
tcaaacaatg tttataatgt
gtatatagaa ttgttcactg taaaaaaaat
ggccaaaatg tgtttttttt
ttaataagta acttgactat aaaataaagc
cgtccgtggg acgactgacc
tcgttgcaaa aaaaaaaaaa aa

ii. HNF4A

Hepatocyte nuclear factor 4 alpha (HNF4alpha, NR2A1, gene symbol HNF4A) is a highly conserved member of the nuclear receptor (NR) superfamily of ligand-dependent transcription factors (Sladeck, et al., Genes Dev., 4(12B): 2353-65 (1990)). HNF4A1 is expressed in liver (hepatocytes), kidney, small intestine, etc. HNF4A2 is the most predominant isoform in the liver. HNF4A regulates most if not all of the apolipoprotein genes in the liver and regulates the expression of many cytochrome P450 genes (e.g., CYP3A4, CYP2D6) and Phase II enzymes and hence may play a role in drug metabolism (Gonzalez, et al., Drug Metab. Pharmacokinet, 23(1):2-7 (2008)).

An exemplary HNF4A gene is represented by NM_178849.2. A nucleic acid encoding HNF4A can include a sequence having at least 80%, 85%, 90%, 95%, 99%, or 100% sequence identity to this sequence or a functional fragment or variant of thereof.

A number of naturally occurring variants of nucleic acids encoding HNF4A and their activities are known in the art. A human hepatocyte nuclear factor 4 gene is described under NCBI GenBank Accession No. BC137539.1.

iii. HNF6A

HNF6 was originally characterized as a transcriptional activator of the liver promoter of the 6-phosphofructo-2-kinase (pfk-2) gene, is expressed in liver, brain, spleen, pancreas, and testis. Lannoy, et al., J. Biol. Chem., 273:13552-13562 (1998). Alternative splicing results in multiple transcript variants.

A Homo sapiens transcript variant mRNA is disclosed under Genbank Accession No. NM_004498.2.

A nucleic acid encoding HNF6 can include a sequence having at least 80%, 85%, 90%, 95%, 99%, or 100% sequence identity to this sequence i.e., the sequence resented by Genbank Accession No. NM_004498.2 or a functional fragment or variant thereof.

A number of naturally occurring variants of nucleic acids encoding HNF6 and their activities are known in the art. A human hepatocyte nuclear factor 6 (HNF6) gene is described under NCBI GenBank Accession No. AF035581. HNF6A is also known as one cut homeobox 1 (ONECUT1).

iv. GATA4

The GATA-binding proteins are a group of structurally related transcription factors that control gene expression and differentiation in a variety of cell types. Members of this family of DNA-binding proteins recognize a consensus sequence known as the ‘GATA’ motif, which is an important cis-element in the promoters of many genes. GATA4 is expressed in adult vertebrate heart, gut epithelium, and gonads. During fetal development, GATA4 is expressed in yolk sac endoderm and cells involved in heart formation. An exemplary GATA4 gene is represented by NM_002052.

v. FOXA2

The FOXA2 gene encodes hepatocyte nuclear factor 3-beta (HNF-3B), also known as forkhead box protein A2 (FOXA2) or transcription factor 3B (TCF-3B). Forkhead box protein A2 is a member of the forkhead class of DNA-binding proteins. These hepatocyte nuclear factors are transcriptional activators for liver-specific genes such as albumin and transthyretin, and they also interact with chromatin. Similar family members in mice have roles in the regulation of metabolism and in the differentiation of the pancreas and liver. The FOXA2 gene is conserved in Rhesus monkey, dog, cow, mouse, rat, chicken, zebrafish, and frog.

An exemplary FOXA2 gene is represented by NM_021784

vi. MYC

MYC (c-Myc) is a regulator gene that codes for a transcription factor, which is multifunctional, nuclear phosphoprotein that plays a role in cell cycle progression, apoptosis and cellular transformation.

In one embodiment, MYC is represented by the sequence.

ctggattttt ttcgggtagt ggaaaaccag
cagcctcccg cgacgatgcc cctcaacgtt
agcttcacca acaggaacta tgacctcgac
tacgactcgg tgcagccgta tttctactgc
gacgaggagg agaacttcta ccagcagcag
cagcagagcg agctgcagcc cccggcgccc
agcgaggata tctggaagaa attcgagctg
ctgcccaccc cgcccctgtc ccctagccgc
cgctccgggc tctgctcgcc ctcctacgtt
gcggtcacac ccttctccct tcggggagac
aacgacggcg gtggcgggag cttctccacg
gccgaccagc tggagatggt gaccgagctg
ctgggaggag acatggtgaa ccagagtttc
atctgcgacc cggacgacga gaccttcatc
aaaaacatca tcatccagga ctgtatgtgg
agcggcttct cggccgccgc caagctcgtc
tcagagaagc tggcctccta ccaggctgcg
cgcaaagaca gcggcagccc gaaccccgcc
cgcggccaca gcgtctgctc cacctccagc
ttgtacctgc aggatctgag cgccgccgcc
tcagagtgca tcgacccctc ggtggtcttc
ccctaccctc tcaacgacag cagctcgccc
aagtcctgcg cctcgcaaga ctccagcgcc
ttctctccgt cctcggattc tctgctctcc
tcgacggagt cctccccgca gggcagcccc
gagcccctgg tgctccatga ggagacaccg
cccaccacca gcagcgactc tgaggaggaa
caagaagatg aggaagaaat cgatgttgtt
tctgtggaaa agaggcaggc tcctggcaaa
aggtcagagt ctggatcacc ttctgctgga
ggccacagca aacctcctca cagcccactg
gtcctcaaga ggtgccacgt ctccacacat
cagcacaact acgcagcgcc tccctccact
cggaaggact atcctgctgc caagagggtc
aagttggaca gtgtcagagt cctgagacag
atcagcaaca accgaaaatg caccagcccc
aggtcctcgg acaccgagga gaatgtcaag
aggcgaacac acaacgtctt ggagcgccag
aggaggaacg agctaaaacg gagctttttt
gccctgcgtg accagatccc ggagttggaa
aacaatgaaa aggcccccaa ggtagttatc
cttaaaaaag ccacagcata catcctgtcc
gtccaagcag aggagcaaaa gctcatttct
gaagaggact tgttgcggaa acgacgagaa
cagttgaaac acaaacttga acagctacgg
aactcttgtg cg

vii. p53 (TP53)

The activity or level of p53 in cells to be reprogrammed is downregulated using any method known in the art. Compositions that can be used to downregulated p53 levels/expression include, but are not limited to antisense oligonucleotides, siRNA, shRNA, miRNA, EGSs, ribozymes, and aptamers, discussed further below. Preferably, p53 gene expression is inhibited using siRNA, shRNA, or miRNA.

In a preferred embodiment, the composition is an siRNA. Examples of oligonucleotides encoding p53 siRNA are 5′-TGACTCCAGTGGTAATCTACTTCAAGAGAGTAGATTACCACTGGA GTCTTTTTTC-3′ and 5′ CGAGAAAAAAGACTCCAGTGGTAATCTACTCTCTTGAAGTAGATT ACCACTGGAGTCA-3′.

B. Vectors encoding Hepatocyte Inducing Factors

The Hepatocyte inducing factors are introduced into a host cell using suitable transformation vectors. Nucleic acids, such as those described above, can be inserted into vectors for expression in cells. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, lentiviruses and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

C. Cells to be Induced

Cells that can be reprogrammed include embryonic stem cells (ESC), induced pluripotent stem cells (iPSC), fibroblast cells, adipose-derived stem cells (ADSC), neural derived stem cells, blood cells. keratinocytes, intestinal epithelial cells and other non-hepatocyte somatic cells. In a preferred embodiment, the non-hepatocyte cell is a fibroblast cell, for example human embryonic fibroblasts (HEFs) or foreskin fibroblasts. The cells are preferably obtained from a mammal, for example, rat, mice, monkeys, dogs, cats, cows, rabbits, horses, pigs, Preferably, the cells are obtained from a human subject.

D. induced Hepatocyte Cells

iHeps are disclosed, which are obtained for example, by a method which includes treating non-hepatocyte cells to overexpress the hepatic fate conversion factors HHEX, HNF4A, HNF6A, GATA4 and FOXA2.

In some embodiments where the cell being induced is not an epithelial cell, iHeps additionally express at least one epithelial cell marker, for example, E-cadherin, and where the cell being induced is a fibroblast, the iHeps obtained following induction of fibroblasts using the methods disclosed herein, do not express the fibroblast marker genes such as COL1A1, and/or THY1 as measured for example by RT-qPCR.

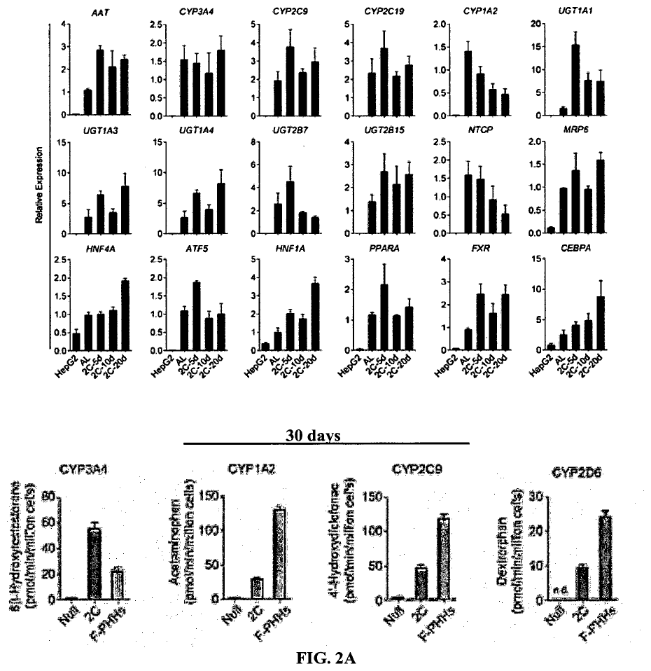

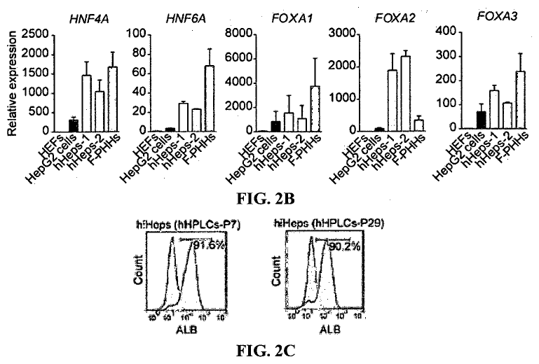

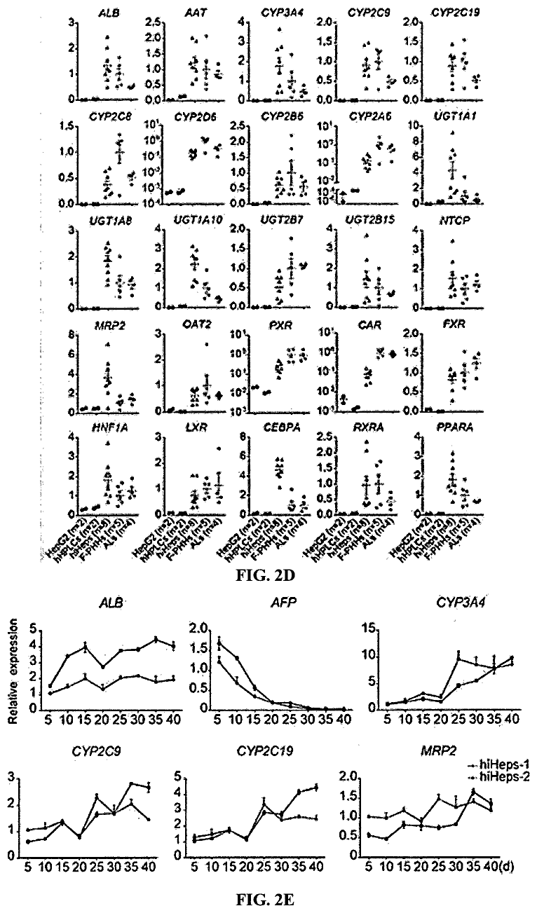

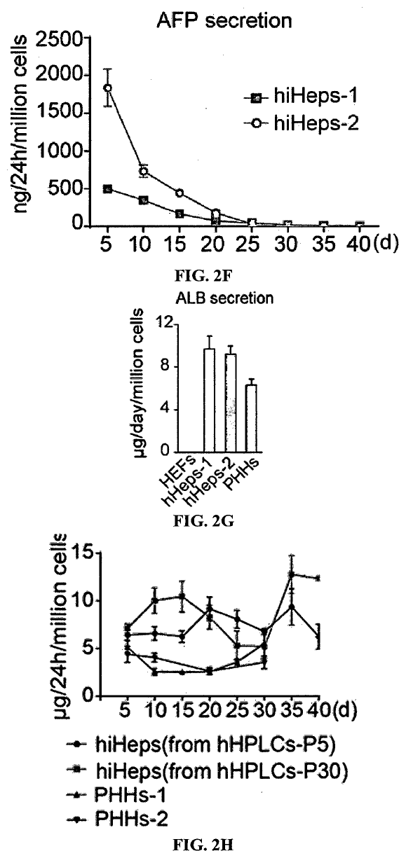

With respect to functional characteristics associated with mature hepatocytes, iHeps possess at least one characteristic selected from the group consisting of: typical hepatocyte-morphology similar to cultured primary hepatocytes from the organism from which the non-hepatocyte cell was obtained. For example, where the non-hepatocyte is a human cell, the iHep has hepatocyte-morphology similar to cultured primary human hepatocytes (PHHs). iHeps are immunopositive for E-cadherin and hepatic-TFs HNF4A, HNF1A, CEBPA, CEBPA. The iHep cells are ALB+ as well, the ALB+ cells in the population of cells obtained following Phase I and Phase II cell culture constitute over 90% of the cell population, as measured by measured by flow cytometry analysis, for example. Second, iHeps show upregulated expression levels of major mature hepatocyte-functional genes when compared with hHPLCs; these expression levels are comparable to those in freshly isolated primary hepatocytes (F-PHHs) and adult liver tissues (ALs) from the organism from which the non-hepatocyte cell was obtained. For example, where the non-hepatocyte is a human cell the expression levels of major mature hepatocyte-functional genes is comparable to expression levels in F-PHHs and adult liver tissues (ALs) (FIGS. 2B and 2D). Adult liver tissue (AL) is a mixture of more than 60% hepatocytes and less than 40% non-hepatocyte such as Kupffer cell, endothelial cells, cholangiocyte, etc. F-PHHs are isolated form AL and typically contain more than 95% hepatocytes. F-PHHs are isolated from AL, following about 2-4 hours of digestion. Both F-PHHs and AL are considered as good controls of hiHeps or any other induced hepatocytes generated in vitro.

Third, the expression of functional genes ALB and CYP450s, are stably maintained for at least 40 days, during which fetal marker AFP is abolished (measured as undetectable levels of AFP secretion in an ELISA assay, RT-qPCR or immunofluorescence staining, for example); other fetal hepatocyte markers including DLK1 and EPCAM are also downregulated in iHeps. Fourth, iHeps express key drug-metabolizing enzymes of CYP450s, UGT1A1 and POR, for example, iHeps are immunopositive for these enzymes. Fifth, iHeps are competent for low-density lipoprotein (LDL) uptake, fatty droplets synthesis and glycogen synthesis. Lastly, ALB secretion by iHeps can be maintained for at least 40 days at a comparable level to that of PHH. These data indicated that hHPLCs gave rise to functional hepatocytes.

Thus, like primary hepatocytes, hiHeps express an additional spectrum of Phase I and II drug-metabolizing enzymes and Phase III drug transporters and albumin. iHeps express at least one drug-metabolizing enzyme selected from the group consisting of CYP3A4, CYP2C9, CYP2C19, CYP2A6, CYP2C8, CYP2D6, CYP2B6, CYP1A2, UGT1A1, UGT1A8, UGT1A10, UGT2B7, UGT2B15 and POR.

The expression levels of at least one of CYP3A4, CYP2B6, CYP2C8, CYP2C9, CYP2C19, CYP2D6, CYP1A2, CYP2A6, UGT1A1 and POR are comparable between iHeps and freshly isolated primary human hepatocytes. In a preferred embodiment, iHeps express CYP3A4, CYP2C9, CYP2C19, CYP2A6, CYP2B6, CYP2C8, CYP2D6, and UGT1A1 and the expression is comparable to levels in freshly isolated primary hepatocytes and/or adult liver tissues. In some preferred embodiments, the expression levels of at least one of CYP3A4, CYP2C9, CYP2C19, CYP2A6, CYP2C8, CYP2D6, CYP2B6, CYP1A2, UGT1A1, UGT1A8, UGT1A10, UGT2B7, UGT2B15, NTCP and POR is superior to the expression level in freshly isolated primary hepatocytes and/or adult liver tissues.

In some embodiments, MYC expression levels in iHeps are lower than the levels found in normal hepatocytes in the corresponding organism as measured for example, by quantitative reverse transcriptase polymerase chain reaction (RT-qPCR), i.e., if the donor organism for the non-hepatocyte cell to be induced is a human subject, the levels are compared to normal hepatocytes found in humans.

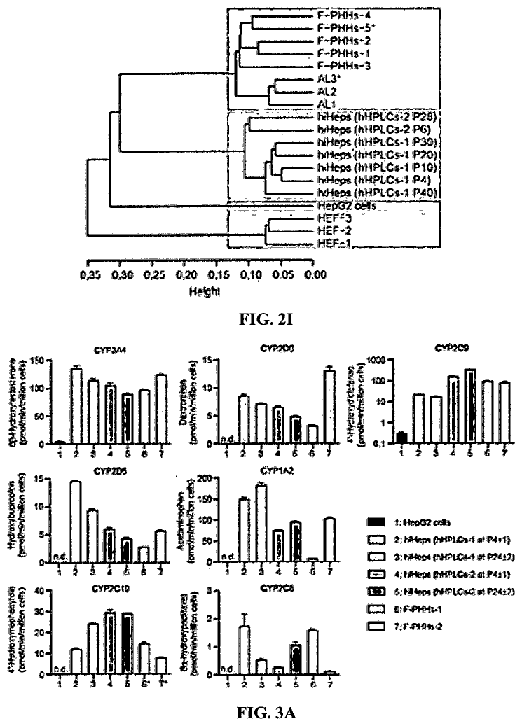

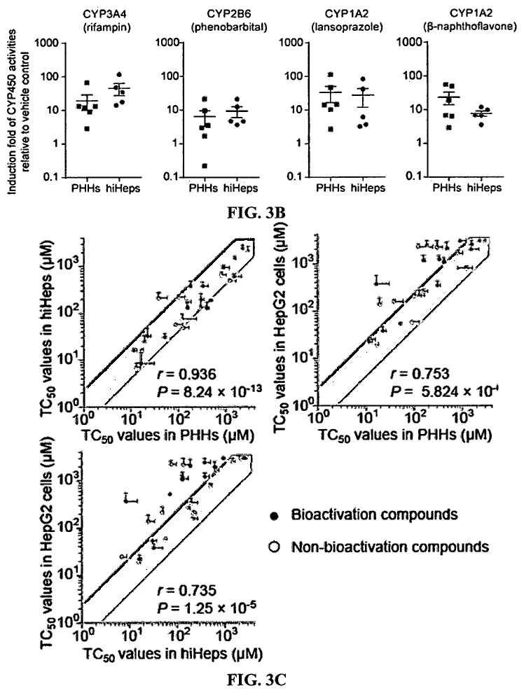

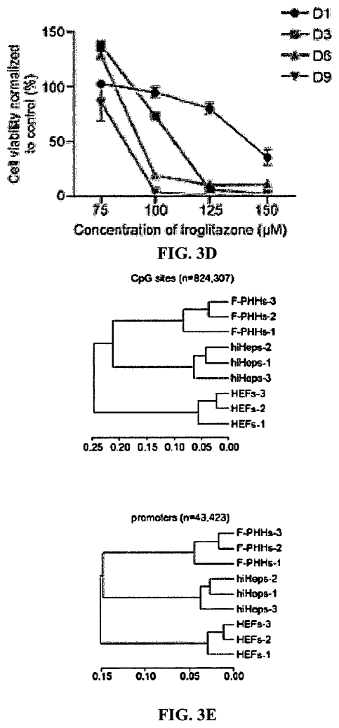

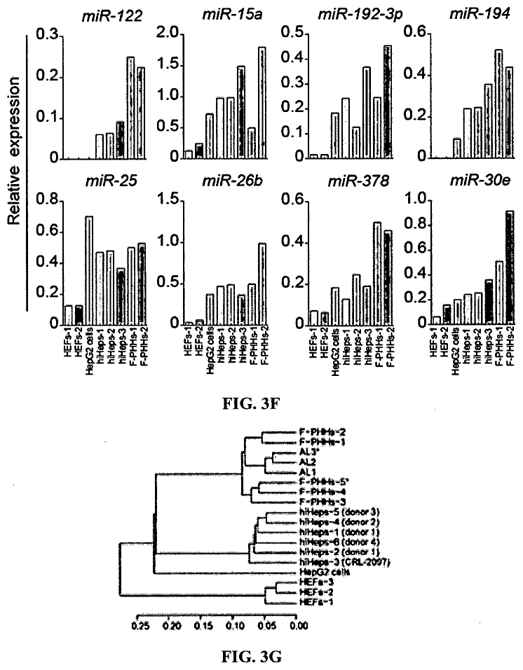

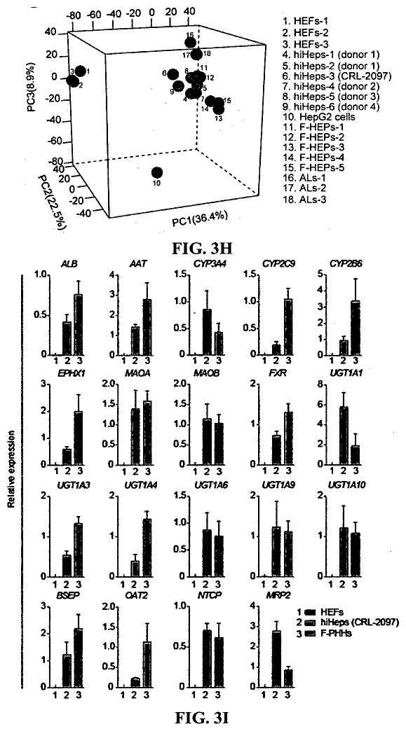

Importantly, the metabolic activities of CYP450s in iHeps are comparable (measured as no statistically significant difference) to the activity in PHHs from the same organism (FIG. 3A).

### III. Method of Making

Various methods disclosed for converting a non-hepatocyte cell into cells with hepatocyte-like properties do not recognize or address problems with limited activation of key hepatic genes and/or low yield of functional cells.

U.S. Patent Application Publication No. 2012/231490 discloses a method of obtaining hepatocytes from iPS cells by introducing, in addition to SOX17, HHEX and HNF4A, one or more genes such as GATA4, GATA6, HNF1A, HNF1B, FOXA1/HNF3A, FOXA2/HNF3B, FOXA3/HNF3G, CEBPA, CEBPB, TBX3 and PROX1. U.S. Patent Application Publication No. 2013/0251694 discloses using exogenous expression cassettes, which may include FOXA2, HNF4A and one or more additional hepatocyte programming factor genes selected from the group consisting of HHEX, HNF1A, FOXA1, TBX3-1, GATA4, NR0B2, SCML1, CEBPB, HLF, HLX, NR1H3, NR1H4, NR1I2, NR3, NR5A2, SEBOX, and ZNF391.

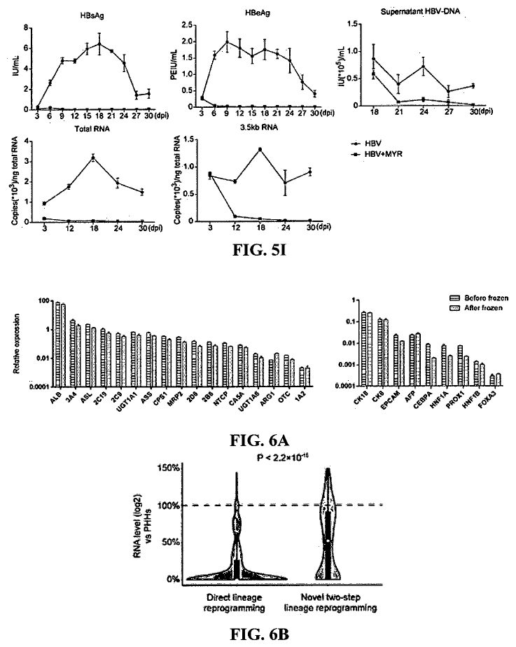

Huang, et al., Nature, 475:386-389 (2011) disclose the direct induction of hepatocyte-like cells from mouse tail-tip fibroblasts by transduction of Gata4, Hnf1α and Foxa3, and inactivation of p19(Arf). Induced cells show typical epithelial morphology. Sekiya and Suzuki, Nature, 475:390-393 (2011)), identified three specific combinations of two transcription factors, Hnf4α plus Foxa1, Foxa2 or Foxa3, that can convert mouse embryonic and adult fibroblasts into cells that resemble hepatocytes in vitro. Previous studies identified a combination of HNF1A, HNF4A, HNF6, ATFS, PROX1 and CEBPA for obtaining hepatocyte-like cells from a non-hepatocyte cell with a one-step induction strategy. Du, et al., Cell Stem Cell, 14:394-403 (2014). However, epigenetic barriers to direct reprogramming such as the packed H3K9me3 heterochromatin domains, exist, making it hard for transcription factors (TFs) to access for activating tissue-specific genes of the target cells, resulting in incomplete cell fate conversion (Becker, et al., Trends in Genetics, 32:29-41 (2016)). Consistently, only limited activation of key hepatic genes located in the H3K9me3 domain in previous direct fibroblast-to-hepatocyte conversion study (FIG. 6B) (See also, Gao, et al., Stem Cell Reports, 9:1813-1824 (2017); Becker, et al., Mol. Cell., 68:1023-1037 e1015 (2017))).

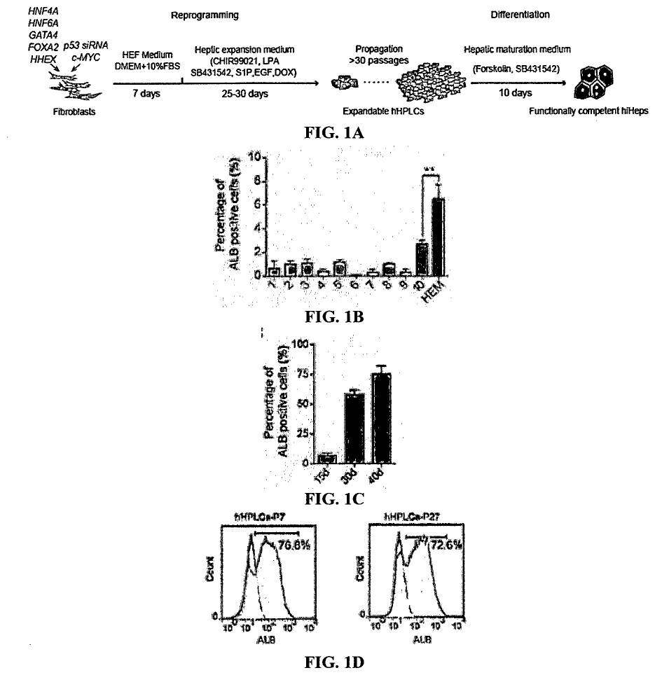

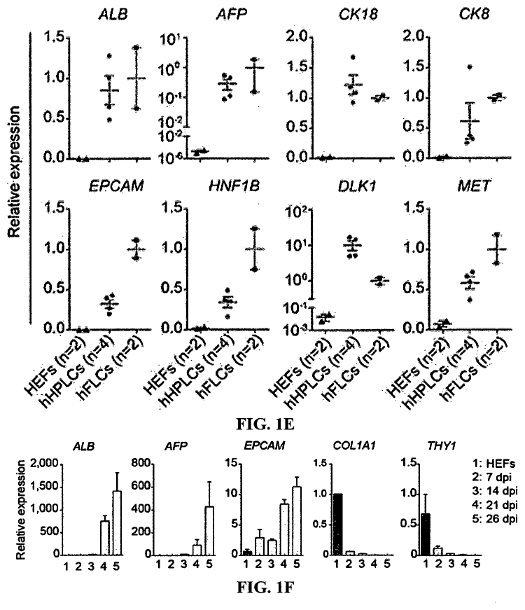

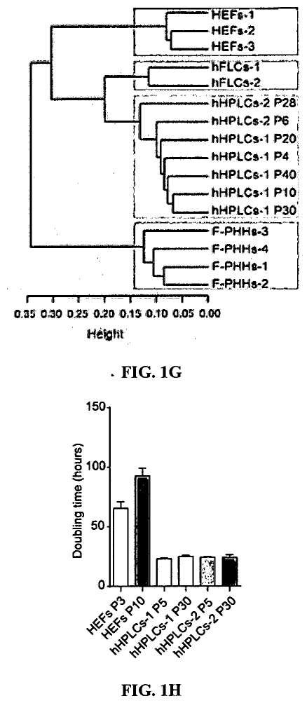

While not being bound by theory, the studies disclosed herein solve this problem, overcoming lineage barriers through an indirect cell-fate conversion route during regeneration. In this system, contrary to the direct fibroblast-hepatocyte conversion (i.e., one-step methods) discussed above, terminally differentiated cells are first de-differentiate into proliferating progenitors and then, by recapitulating certain developmental programs, re-differentiate into highly competent functional cells along the epigenetic landscape in response to various differentiation signals. This is based on two principles, i.e. progenitor cells with relatively open chromatin architectures are more amenable for accurate cell-fate induction and proliferation of such cells allows the generation of abundant functional cells. The methods disclosed herein therefore explored a new two-step strategy to generate functionally competent human hepatocytes by introducing a plastic intermediate stage of expandable proliferative progenitor cells into lineage reprogramming (FIG. 1A).

In the methods disclosed herein, the non-hepatocyte is reprogrammed into an iHep by upregulating hepatocyte inducing factors in the cell, in combination with upregulating MYC and downregulating p53, and culturing the cells for a sufficient period of time as disclosed herein to convert the cell into a cell termed hepatic progenitor-like cell (HPLC), and subsequently, into a cell with hepatocyte-like properties (iHep). The non-hepatocyte cells to be induced are obtained from the donor animal using methods known in the art.

The reprograming method includes two phases, a hepatic progenitor cell generation phase (Phase I) and induced hepatocyte (iHep) generation phase (Phase II). Phase I is a reprogramming phase, which includes the following steps: (a) upregulating hepatocyte inducing factors in the non-hepatocyte cell to obtain transformed cells and culturing the cells in cell culture medium (transformation phase) and (b) replating and culturing the transformed cells in HEM (Hepatic Expansion Medium) (expansion phase). Phase II includes culturing the cells in a custom differentiation medium with at least one cAMP agonist/cAMP analogue and a TGFβ receptor inhibitor (maturation phase). A schematic for the disclosed method is shown in FIG. 1A.

A. Phase I

In the first step of Phase I(a), the cells are treated to upregulate/overexpress hepatocyte inducing factors HHEX, HNF4A, HNF6A, GATA4, and FOXA2. Preferably, the cells are additionally treated to upregulate/overexpress MYC and/or downregulate p53. In some embodiments, the cells are not treated in Phase I to upregulate/overexpress SOX 17, HNF1A, FOXA1, TBX3-1, NR0B2, SCML1, CEBPB, HLF, HLX, NR1H3, NR1H4, NR1I2, NR1I3, NR5A2, SEBOX, ZNF391, ATFS, PROX1, HNF1B, FOXA1/HNF3A, FOXA2/HNF3B, FOXA3/HNF3G, CEBPA, or TBX3.

Treatment to upregulate/overexpress hepatocyte inducing factors preferably includes introducing genes encoding HHEX, HNF4A, HNF6A, GATA4, FOXA2 and MYC and an oligonucleotide encoding p53 siRNA into cells to obtain transfected cells, using any method known in the art for introducing genes into a cell. Preferably, the genes are introduced using expression systems such as adenovirus, lentivirus etc, which are known in the art. Lentivirus expression systems are exemplified herein.

Thus, transformed cells resulting from Phase I(a) can be obtained by transfecting cells as disclosed herein or any other non-transfection method in the art to upregulate expression of the genes of interest, for example, treatment with small molecules.

(i) HEF Cell Culture

In Phase I(a), the cells transfected/treated to overexpress HHEX, HNF4A, HNF6A, GATA4, FOXA2 and MYC and downregulate p53 (herein, transformed cells), are cultured in conventional cell culture medium, for example, FIEF (human embryonic fibroblast) medium for 5-10 days, preferably, for at least 7 days in this first step, for example, 7, 8, 9 or 10 days. Most preferably, the cells are maintained in HEF medium for about 7 days. An exemplary HEF medium is Dulbecco's modified Eagle's medium (DMEM) containing 10% fetal bovine serum (FBS), 1% GlutaMAX, 1% Non-Essential Amino Acids (NEAA) and 1% penicillin/streptomycin (PS).

(ii) Transformed Cells Cultured in HEM

The transformed cells are then replated and expanded in HEM (which is a supplemented hepatocyte maintenance/expansion medium) for a period of about 15 to 40 days, preferably for about 20-30 days, and more preferably, for about 20-25 days (expansion phase). In a preferred embodiment, the transformed cells are obtained by transfection and are treated with a suitable agent to enrich for transfected cells, for example, puromycin, for about 24 hrs prior to replating. Puromycin is an antibiotic. The infected cells are resistant to the puromycin, for there are resistance genes in the vectors

A preferred HEM, is shown in Table 2B.

The M10 medium is DMEM/F12 supplemented with epidermal growth factor (EGF), a glycogen synthase kinase 3 inhibitor (CHIR99021), transforming growth factor β receptor inhibitor (E-616452), lysophosphatidic acid (LPA), sphingosine 1-phosphate (SIP); insulin-transferrin-sodium selenite (ITS), nicotinamide and 2-phospho-L-ascorbic acid (pVc) (Table 2A and 2B). By contrast, in a particularly preferred embodiment, HEM includes 50% DMEM/F12, 50% William's E Medium supplemented with an antibiotic such 1% PS [[Penicillin and Streptomycin]], 2% B27 (without VA), 5 mM Nicotinamide, 200 μM 2-phospho-L-ascorbic acid, 3 μM CHIR99021, 5 μM SB431542, 0.5 μM Sphingosine-1-phosphate (SIP), 5 μM lysophosphatidic acid (LPA) and 40 ng/ml epidermal growth factor (EGF).

The preferred GSK inhibitor is the aminopyrimidine, CHIR99021 having the chemical name [6-[[2-[[4-(2,4-Dichlorophenyl)-5-(5-methyl-1H-imidazol-2-yl)-2-pyrimidinyl]amino]ethyl]amino]-3-pyridinecarbonitrile], used in a concentration of about 3 Other GSK inhibitors can also be used in the methods disclosed herein, and they include, but are not limited to BIO-acetoxime (for example 1 μM); GSK 3I inhibitor XV; SB-216763; CHIR 99021 trihydrochloride, which is the hydrochloride salt of CHIR99021; GSK-3 Inhibitor IX [((2Z,3E)-6′-bromo-3-(hydroxyimino)-[2,3′-biindolinylidene]-2′-one]; GSK 3 IX [6-Bromoindirubin-3′-oxime]; GSK-3β Inhibitor XII [3-[[6-(3-Aminophenyl)-7H-pyrrolo[2,3-d]pyrimidin-4-yl]oxy]phenol]; GSK-3 Inhibitor XVI [6-(2-(4-(2,4-dichlorophenyl)-5-(4-methyl-1H-imidazol-2-yl)-pyrimidin-2-ylamino)ethyl-amino)-nicotinonitrile]; SB-415286 [3-[(3-chloro-4-hydroxyphenyl)amino]-4-(2-nitrophenyl)-1H-pyrrole-2,5-dione]; and Bio [(2′Z,3′E)-6-bromoindirubin-3′-oxime].

The TGFβ inhibitor preferably inhibits the TGFβ type 1 receptor activing receptor-like kinase (ALK) 5 in some embodiments, and can additionally inhibit ALK 4 and the nodal type receptor 1 receptor ALK7 in other embodiments. The TGFβ receptor inhibitor can be SB431542 (4-[4-(1,3-benzodioxol-5-yl)-5-(2-pyridinyl)-1H-imidazol-2-yl]benzamide); E-616452 ([2-(3-(6-Methylpyridin-2-yl)-1H-pyrazol-4-yl)-1,5-naphthyridine]; or other TGFβ inhibitors which are known in the art and are commercially available. Examples include A83-01 [3-(6-Methyl-2-pyridinyl)-N-phenyl-4-(4-quinolinyl)-1H-pyrazole-1-carbothioamide]; SB 505124 [2-[4-(1,3-Benzodioxol-5-yl)-2-(1,1-dimethylethyl)-1H-imidazol-5-yl]-6-methyl-pyridine]; GW 788388 [4-[4-[3-(2-Pyridinyl)-1H-pyrazol-4-yl]-2-pyridinyl]-N-(tetrahydro-2H-pyran-4-yl)-benzamide]; and SB 525334 [6-[2-(1,1-Dimethylethyl)-5-(6-methyl-2-pyridinyl)-1H-imidazol-4-yl]quinoxaline], and dorsomorphine.

In a particularly preferred embodiment, the combination of supplements, small molecules and growth factors are selected to provide a yield of HPLCs that is more than the yield of HPLCs obtained using M10 for the same culture time. Yield of HPLCs at any given time point can be measured as a percent of ALB⁺ cells at the end of Phase I. Preferably, the selected combination of supplements, small molecules and growth factors provides over a 2 fold increase in yield of ALB⁺ cells, for example, between at least 2 and 30 fold increase in yield, preferably, at least a 10-30 fold increase in yield and even more preferably, between at least 20 and 30 fold increase in yield. For example, the increase in yield can be 21, 22, 23, 24, 25, 26, 27, 28, 29, or 30 fold. The preferred combination of basal medium, supplements, small molecules and growth factors are shown in Table 2B, which as exemplified in this application provides an increased yield of ALB⁺ cells of over 200% (6.5% ALB⁺ cells) at day 15, when compared to yield with M10 medium (2.7% ALB⁺ cells). The SB431542 is used at a concentration between 1 and 10 μM, more preferably, between 1 and 7 μM and even more preferably, between 3 and 7 μM and most preferably, at about 5 μM.

Methods for determining the percent of ALB+ cells are known in the art and exemplified herein using a combination of immunofluorescence.

The end of Phase I is optionally followed by propagation/passaging, following which the cells are subjected to Phase II, by culturing the cells from Phase I in a custom differentiation medium.

B. Phase II

Cells harvest from Phase I are cultured in HEM medium until confluent and they were further cultured in medium supplemented with at least one cAMP agonist and a TGFβ receptor inhibitor, preferably, SB431542 (2C medium) for a period of at least 5 days, preferably between 5 and 40 days, for example for 5, 6, 7, 8, 9, 10, 11, 12, 13, 14 or 15 days, and up to 40 days following which induced hepatocytes are harvested. These time periods are not limiting, as cells can be cultured in 2C medium for longer periods so long they remain viable (i.e., they do not die) for further applications.

The preferred cAMP agonist is Forskolin. However, any cAMP agonist can be used. Examples include, but are not limited to prostaglandin E2 (PGE2), rolipram, genistein and cAMP analogs such as dbcAMP or 8-bromo-cAMP. The cAMP agonist is used at a concentration between 30 and more preferably, between 40 and 60 μM and even more preferably, between 45 and 55 μM.

The SB431542 is used at a concentration between 5 and 20 more preferably, between 5 and 15 μM and even more preferably, between 10 and 15 μM and most preferably, at about 10 μM.

Examples of basal cell culture medium for hepatocytes that can be used to make the 2C medium include, but are not limited to HCM (hepatocyte culture medium); William's E medium containing 2% B27 (Gibco), 1% GlutaMAX; RPMI 1640; Dulbecco's Modified Eagle Medium; Dulbecco's Modified Eagle Medium/Nutrient Mixture F-12 Iscove's Modified Dulbecco's Medium. In a particularly preferred embodiment, 2C medium is HCM supplemented with 50 μM forskolin or 50 μM dbcAMP and 10 μM SB431542.

The method optionally includes a step of (a) confirming that the non-hepatocyte cell has acquired hepatocyte-progenitor-like properties after Phase I, and (b) confirming that the non-hepatocytes have acquired hepatocyte-like properties after Phase II, using morphological and functional characteristics as well as gene expression.

Morphological confirmation methods include the confirmation of morphological characteristics specific for hepatocyte progenitor cells-like (HPLCs) and mature hepatocyte-like (iHEP).

HPLCs can be identified based on upregulation of hHPC-enriched genes, including those with known roles for hHPCs, for example, ALB, AFP, EPCAM, CK8, CK18, HNF1B, DLK1, and MET.

Treated cells can be identified as induced hepatocytes using one or more of the following characteristics: (i) immunopositive for E-cadherin and hepatic-TFs HNF4A, HNF1A, CEBPA, CEBPB; (ii) the expression levels of major mature hepatocyte-functional genes were found dramatically upregulated in hiHeps compared with hHPLCs, and were comparable to those in F-PHHs and adult liver tissues (ALs); (iii) their ability to express ALB at a level comparable to that of primary human hepatocytes, preferably, the expression of functional genes ALB and CYP450s, were stably maintained for at least 40 days, preferably at a level comparable to PHHs during which fetal marker AFP was abolished. Other fetal hepatocyte markers including DLK1 and EPCAM were also downregulated in hiHeps; (iv) expression of one or more of the major cytochrome P450 enzymes, CYP3A4, CYP1A2, CYP2A6, CYP2B6, CYP2C8, CYP2D6, CYP2C9, and CYP2C19; expression of phase II enzyme or phase II transporter selected from the group consisting of UGT1A1, POR, UGT1A3, UGT1A4, UGT1A6, UGT1A9, UGT2B7, UGT2515, NTCP, MRP6, MRP2, FMO5, MAOA, MAOB, EPHX1; (v) competence for low-density lipoprotein (LDL) uptake, fatty droplets synthesis and glycogen synthesis. Successful induction can be confirmed by the presence of an epithelial marker and the absence of a marker for the cell, which is being induced. For example, where the cell being induced is a fibroblast, additional indication that the cells has been induced into a hepatocyte-like cell can be expression of at least one epithelial cell marker, for example, E-cadherin, and absence of expression of the fibroblast marker genes such as COL1A1, THY1 and α-fetoprotein as measured for example by RT-qPCR.

A. Upregulating Hepatocyte Inducing Factors and MYC

Hepatocyte inducing factors and MYC are upregulated by contacting the non-hepatocyte with factors which upregulate gene expression and or protein levels/activity of the Hepatocyte inducing Factors and MYC. These facts include, but are not limited to nucleic acids, proteins and small molecules.

For example, upregulation may be accomplished by exogenously introducing the nucleic acids encoding the hepatocyte inducing Factor(s) and optionally, MYC, into the non-hepatocyte (host cell). The nucleic acid may be homologous or heterologous. The nucleic acid molecule can be DNA or RNA, preferably, mRNA. Preferably, the nucleic acid molecule is introduced into the non-hepatocyte cell by lentiviral expression.

The host cell is transformed to overexpress hepatocyte inducing HHEX, HNF4A, GATA4, HNF6A, and FOXA2. Preferably, the cell is additionally transformed overexpress the proliferation factor MYC. Vectors containing nucleic acids to be expressed can be transferred into host cells. Nucleic acids can be transfected into mammalian cells by techniques including, for example, calcium phosphate co-precipitation, DEAE-dextran-mediated transfection, lipofection, electroporation, or microinjection. The ex vivo methods disclosed herein can include, for example, the steps of harvesting cells from a subject/donor, culturing the cells, transducing them with an expression vector, and maintaining the cells under conditions suitable for expression of the encoded polypeptides. These methods are known in the art of molecular biology.

Upregulation may also be accomplished by treating the cells with factors known to increase expression of genes encoding the hepatocyte inducing factors/MYC and/or factors known to increase the corresponding protein levels. For example, Zhao, et al., Cell Res., 23(1):157-161 (2013), disclose a method for promoting the emergence of PROX1 and HNF6-expressing cells from hESCs using the induction factors FGF7, BMP2 and BMP4. Known factors, including small molecules and/or proteins which upregulate hepatocyte inducing factors gene expression or protein levels can also be used.

B. Downregulating p53

p53 can be downregulated by treating cells to downregulate p53 gene expression, mRNA levels or protein levels. This step includes contacting the cells with known any molecule that is known to downregulate p53 gene expression, mRNA or protein levels, including but not limited to nucleic acid molecules, small molecules and protein.

p53 gene expression can be inhibited using a functional nucleic acid, or vector encoding the same, selected from the group consisting of antisense oligonucleotides, siRNA, shRNA, miRNA, EGSs, ribozymes, and aptamers. Preferably, p53 gene expression is inhibited using siRNA, shRNA, or miRNA.

1. RNA Interference

In some embodiments, P53 gene expression is inhibited through RNA interference. Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point, the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of RNAi or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

2. Antisense

p53 gene expression can be inhibited using can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

An “antisense” nucleic acid sequence (antisense oligonucleotide) can include a nucleotide sequence that is complementary to a “sense” nucleic acid encoding a protein, e.g., complementary to the coding strand of a double-stranded cDNA molecule or complementary to the p53 encoding mRNA. Antisense nucleic acid sequences and delivery methods are well known in the art (Goodchild, Curr. Opin. Mol. Ther., 6(2):120-128 (2004); Clawson, et al., Gene Ther., 11(17):1331-1341 (2004). The antisense nucleic acid can be complementary to an entire coding strand of a target sequence, or to only a portion thereof. An antisense oligonucleotide can be, for example, about 7, 10, 15, 20, 25, 30, 35, 40, 45, 50, 55, 60, 65, 70, 75, 80, or more nucleotides in length.

An antisense nucleic acid sequence can be designed such that it is complementary to the entire p53 mRNA sequence, but can also be an oligonucleotide that is antisense to only a portion of the p53 mRNA. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Other examples of useful antisense oligonucleotides include an alpha-anomeric nucleic acid. An alpha-anomeric nucleic acid molecule forms specific double-stranded hybrids with complementary RNA in which, contrary to the usual beta-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

3. Aptamers

In some embodiments, the inhibitory molecule is an Aptamer. Aptamers are molecules that interact with a target molecule, preferably in a specific way. Aptamers can bind the target molecule with a very high degree of specificity. For example, aptamers have been isolated that have greater than a 10,000 fold difference in binding affinities between the target molecule and another molecule that differ at only a single position on the molecule. Because of their tight binding properties, and because the surface features of aptamer targets frequently correspond to functionally relevant parts of the protein target, aptamers can be potent biological antagonists. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

4. Ribozymes

p53 gene expression can be inhibited using ribozymes. Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

5. Triplex Forming Oligonucleotides

p53 gene expression can be inhibited using triplex forming molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

6. External Guide Sequences

p53 expression can be inhibited using external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

7. ShRNA

p53 expression can be inhibited using small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides (Brummelkamp et al., Science 296:550-553 (2002); Lee et al., Nature Biotechnol. 20:500-505 (2002); Miyagishi and Taira, Nature Biotechnol. 20:497-500 (2002); Paddison et al., Genes Dev. 16:948-958 (2002); Paul et al., Nature Biotechnol. 20:505-508 (2002); Sui (2002) supra; Yu et al., Proc. Natl. Acad. Sci. USA 99(9):6047-6052 (2002).

C. Delivery Vehicles

Methods of making and using vectors for in vivo expression of functional nucleic acids such as antisense oligonucleotides, siRNA, shRNA, miRNA, EGSs, ribozymes, and aptamers are known in the art.

For example, the delivery vehicle can be a viral vector, for example a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome. The recombinant retrovirus can then be used to infect and thereby deliver to the infected cells nucleic acid encoding the hepatocyte inducing factor(s). The exact method of introducing the altered nucleic acid into the host cell is, of course, not limited to the use of retroviral vectors. Other techniques are widely available for this procedure including the use of adenoviral vectors, adeno-associated viral (AAV) vectors, lentiviral vectors, pseudotyped retroviral vectors, and others described in (Soofiyani, et al., Advanced Pharmaceutical Bulletin, 3(2):249-255 (2013). Viruses can be modified to enhance safety, increase specific uptake, and improve efficiency (see, for example, Zhang, et al., Chinese J Cancer Res., 30(3):182-8 (2011), Miller, et al., FASEB J, 9(2):190-9 (1995), Verma, et al., Annu Rev Biochem., 74:711-38 (2005)).

Physical transduction techniques can also be used, such as liposome delivery and receptor-mediated and other endocytosis mechanisms (see, for example, Schwartzenberger et al., Blood, 87:472-478 (1996)). Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, nucleic acid or vectors encoding the hepatocyte inducing factors can be delivered in vivo by electroporation as well as by means of a sonoporation. During electroporation electric pulses are applied across the cell membrane to create a transmembrane potential difference, allowing transient membrane permeation and transfection of nucleic acids through the destabilized membrane (Soofiyani, et al., Advanced Pharmaceutical Bulletin, 3(2):249-255 (2013)). Sonoporation combines the local application of ultrasound waves and the intravascular or intratis sue administration of gas microbubbles to transiently increase the permeability of vessels and tissues (Escoffre, et al., Curr Gene Ther., 13(1):2-14 (2013)). Electroporation and ultrasound based techniques are targeted transfection methods because the electric pulse or ultrasound waves can be focused on a target tissue or organ and hence gene delivery and expression should be limited to thereto. Expression or overexpression of the disclosed hepatocyte inducing factors accomplished with any of these or other commonly used gene transfer methods, including, but not limited to hydrodynamic injection, use of a gene gun.

### IV. Methods of Using

The studies disclosed herein show that human hepatocytes with drug metabolic function can be generated by lineage reprogramming, thus providing a cell resource for pharmaceutical applications.

A. In Vitro and Research Applications

(i) Drug Testing

Liver parenchymal cells play a key role in drug development because the liver plays a central role in the metabolic activity of the drug. At present, the main cause of failure of a drug candidate is its ADME (absorption, distribution, metabolism, excretion) is not ideal. An essential part of drug discovery research is to the metabolic and toxicological effects of the candidate drug on liver cells, human liver parenchymal cells with full participation of drug metabolism. Currently the main hepatocytes used for in vitro drug development are human adult primary hepatocytes. Due to their limited sources, and the difficulty of maintaining primary hepatocyte function in vitro, their application in drug development is quite limited.

hiHeps disclosed herein which express drug-metabolizing enzymes can be used in vitro drug metabolism studies.

(ii) Research

The problem encountered in studies involving infectious diseases is the lack of adequate animal models. Both hHPLCs and hiHeps can be used to construct humanized mouse models for study of infectious diseases, for example, hepatitis B and C infections. These animal models can provide a reliable in vivo platform for in the development of vaccines and drugs for treating infectious diseases, particularly diseases that infect the liver.

iHeps can serve as an in vitro model to recapitulate HBV infection. hiHeps express the HBV receptor NTCP in hiHeps. HBV-infected hiHeps were immunopositive for HBcAg. Analysis of the secretion of HBsAg and HBeAg, and the expression of HBV-DNA, -RNA, -cccDNA in hiHeps show that HBV markers in hiHeps are comparable to those in PHHs. Secretion of HBsAg and HBeAg gradually increased and peaks at 20 dpi; the supernatant and intracellular HBV-DNA retained their expression for at least 36 days Collectively, this indicates that hiHeps obtained according to the methods disclosed herein can support long-term HBV infection in vitro.

B. In Vivo Applications

Liver failure and loss of function is one of the most severe consequences of liver disease. Because of its rapid onset, rapid progression, liver transplantation is the primary means of treatment of these diseases. However, donor scarcity presents a serious lack of many patients dying while waiting for liver transplantation.

Thus, iHeps can be used in the treatment of liver failure and loss of function diseases, for example.

Transplanting isolated iHeps or HPLCs by percutaneous or transjugular infusion into the portal vein, or injecting into the splenic pulp or the peritoneal cavity, is a less invasive procedure compared with liver transplantation. The iHeps are preferably obtained from the same animal being treated. As the host liver is not removed or resected, the loss of graft function should not worsen liver function. Furthermore, isolated iHeps could be, potentially, cryopreserved for ready access. The iHeps can be used as a vehicle for ex vivo gene therapy for example, for rescuing patients from radiation-induced liver damage resulting from radiotherapy for liver tumors. iHeps can be transplanted into a recipient organism using a carrier such as a matrix that known for transplantation of hepatocytes. For example, Zhou, et al., Liver Transpl., 17(4):418-27 (2011) discloses the use of decellularized liver matrix (DLM) as a carrier for hepatocyte transplantation. Schwartz, et al., Int. J. Gastroentrol., 10(1): discloses isolating liver and pancreas cells from tissue samples, seeded onto a poly-L-lactic acid matrix and re-implanted into the mesentery of the same patient.

iHeps can also be used in the bio-artificial liver support systems. Bioartificial liver support system based on the disclosed cells are constructed to temporarily replace the main function of liver failure (remove of hazardous substances, provide the liver synthetic biologically active substances), to stabilize and improve the patient's internal environment, until a suitable donor source for transplantation. Methods for making bioartifical liver are disclosed for example in U.S. Publication No. 2008/0206733.

### V. Kits

Kits for inducing in vitro reprograming of non-hepatocytes into induced hepatocytes with functional hepatocyte metabolic properties are disclosed. The kit includes factors which up regulate hepatocyte inducing factors HHEX, HNF4A, HNF6A, GATA4, FOXA2, MYC and factors which downregulate p53 gene expression and/or protein activity. In one embodiment, the kit includes any DNA sequence of HHEX, HNF4A, HNF6A, GATA4, and FOXA2, MYC and DNA sequence to downregulate p53 gene expression. In a preferred embodiment, the kit includes lentiviruses which overexpress HHEX, HNF4A, HNF6A, GATA4, and FOXA2, MYC gene and nuclei acid which inhibits p53 gene expression.

### Examples

To generate human hepatic progenitor cells (hHPCs) from human embryonic fibroblasts (HEFs), several candidate TFs for screening were identified based on (i) their importance in hepatic organogenesis and (ii) computational analysis of our RNA-sequencing data of human fetal liver cells (hFLCs), specified human hepatic progenitors that originate hepatocytes (TABLE 1).

TABLE 1
Candidate TFs for screening

| Number | Gene symbol | Accession |
|---|---|---|
| 1 | FOXA2 | NM_021784 |
| 2 | HHEX | NM_002729 |
| 3 | GATA4 | NM_002052 |
| 4 | ONECUT1 | NM_004498 |
| 5 | HNF4A | NM_178849 |
| 6 | HNF1B | NM_000458 |
| 7 | NR5A2 | NM_205860 |
| 8 | PROX1 | NM_001270616 |
| 9 | ONECUT2 | NM_004852 |
| 10 | FOXA3 | NM_004497 |
| 11 | USF2 | NM_003367 |
| 12 | HLF | NM_002126 |
| 13 | USF1 | NM_007122 |
| 14 | SHP | NM_021969 |
| 15 | CEBPB | NM_005194 |

Various candidate TF-combinations, coupled with c-MYC and P53 small interfering RNAs (P53 siRNAs) were screened in order to overcome proliferation arrest and cell death (Du, et al., Cell Stem Cell, 16:119-134 (2015).

### Materials and Methods

Human Primary Cell Isolation and Culture

The present study was approved by the Clinical Research Ethics Committee of China-Japan Friendship Hospital (Ethical approval No: 2009-50) and Stem Cell Research Oversight of Peking University (SCRO201103-03), and it was conducted according to the principles of the Declaration of Helsinki. Human embryonic skin and fetal liver tissues at 14 gestational weeks were obtained from aborted tissue with informed patient consent. Human embryonic skin tissues were minced with forceps and incubated in 1 mg/ml collagenase IV (Gibco) for 1-2 hours at 37° C. After enzyme treatment, cells were collected by centrifugation and resuspended in HEF medium (Dulbecco's modified Eagle's medium (DMEM, Gibco) containing 10% fetal bovine serum (FBS, Ausbian), 1% GiutaMAX (Gibco), 1% Non-Essential Amino Acids (NEAA, Gibco) and 1% penicillin/streptomycin (PS, Gibco)). Cells were plated on 10 cm tissue culture dishes and grown in HEF medium.

Fetal liver cells were obtained as previously described (Lilja et al., Transplantation 64, 1240-1248 (1997). Briefly, fetal liver tissue was cut into 1-3 mm3 fragments for digestion in 10 ml of RPMI 1640 medium supplemented with 1 mg/ml collagenase IV. Digestion was performed at 37° C. for 15-20 minutes, and erythrocytes were eliminated by low-speed centrifugation. Cells were washed 3 times with RPMI 1640 medium and collected by centrifugation.

Human primary hepatocytes were isolated from human donor livers not used for liver transplantation after receiving informed consent (Seglen, Preparation of isolated rat liver cells. Methods in cell biology 13, 29-83 (1976). Briefly, liver tissues were perfused with collagenase IV and dispase (Sigma-Aldrich) until the tissue was incompact and separated with tweezers. Hepatocytes were washed 3 times with HCM (Lonza), plated in collagen-coated plates and cultured in HCM. For the long-term PHH experiment, PHHs were cultured in 2C medium (the basal medium was HCM or William's E medium containing 2% B27 (Gibco), 1% GlutaMAX, and cAMP signaling activators (50 μM forskolin or 50 μM dbcAMP) and 10 μM SB431542 were added). For sandwich culture experiment, PHHs were plated and then overlaid with ice-cold 0.25 mg/ml Matrigel (BD Biosciences) in DMEM supplemented with 1% ITS (Gibco), 1% GlutaMAX, 1% NEAA, 1% PS and 10-7 M dexamethasone for 24 hours. Cultures and further experiments were all processed in this medium.

HepG2 cell was a gift from Hui Zhuang (Peking University Health Science Center) and cultured in DMEM containing 10% FBS, 1% GlutaMAX, 1% PS and 1% NEAA (Gibco).

### Molecular Cloning and Lentivirus Production

Complementary DNA of the transcription factors was amplified from human full-length TrueClones™ (Origene) and inserted into pCDH-EF1-MCS-T2A-Puro (System Biosciences) according to the user's manual. c-MYC was cloned into a inducible system of Fu-tet-hOct4 (hOct4 was replaced by c-MYC) (Hou et al., Science 341, 651-654 (2013)). The oligonucleotides encoding p53 siRNA were 5′-TGACTCCAGTGGTAATCTACTTCAAGAGAGTAGATTACCACTGGAG TCTTTTTTC-3′ and 5′ TCGAGAAAAAAGACTCCAGTGGTAATCTACTCTCTTGAAGTAGATTACCACTGG AGTCA-3′. The oligonucleotides were ligated downstream of the U6 promoter in a Lenti-Lox3.7 (pLL3.7) vector (Obach, et al., Drug metab Dispos., 27(11): 1350-9 (1999)). Lentivirus production and collection were described previously (Obach, et al., Drug metab Dispos., 27(11): 1350-9 (1999)).

Generation of hHPLCs and hiHeps

Human fibroblasts were infected with lentivirus containing the 5 transcription factors, Fu-tet-c-MYC, FUdeltaGW-rtTA and P53 siRNA at the m.o.i of 10-20 in HEF medium containing 10 μg/ml polybrene for 12 hours. Cells were washed with PBS and cultured in HEF medium for 7 days. The infected cells were treated with 2 μg/ml puromycin for 24 hours and were then replated every 5 days for 4-5 times in HEM (50% DMEM/F12, 50% E Medium supplemented with 1% PS, 2% B27 (without VA), 5 mM Nicotinamide, 200 μM 2-phospho-L-ascorbic acid (pVc), 3 μM CHIR99021, 5 μM SB431542, 0.5 μM Sphingosine-1-phosphate (S1P), 5 μM Lysophosphatidic acid (LPA), 40 ng/ml EGF and 2 μg/ml doxycycline) until all cells converted into an epithelial morphology. hHPLCs were maintained in HEM and passaged every 4 days at a ratio of 1:5. The 10 different media tested for hepatic progenitor maintenance or expansion are listed in Table 2A.

TABLE 2A
Media tested for hepatic progenitor maintenance or expansion

| | Basal | | |
|---|---|---|---|
| Num. | medium | Key components | Reference |
| 1 | WEM | ITS, BSA, Nicotinamide, NaHCO₃, | Lazaro et al, |
| | | 2-phospho-l-ascorbic acid, Glucose, | 2003 |
| | | GlutaMAX, EGF, Dexamethasone | |
| 2 | DMEM/F12 | ITS, EGF, BSA, β-Mercaptoethanol, | Kubota and |
| | | GlutaMAX, Dexamethasone, | Reid. 2000 |
| | | Nicotinamide | |
| 3 | DMEM/F12 | N2, B27, EGF, HGF, bFGF, | Huch et al. |
| | | CHIR99021, A-83-01, Nicotinamide, | 2015 |
| | | Forskolin | |
| 4 | DMEM/F12 | FBS, ITS, EGF, HGF, | Yu, et al |
| | | β-Mercaptoethanol, Dexamethasone, | 2013 |
| | | Nicotinamide, | |
| 5 | DMEM/F12 | BSA, ITS, EGF, Dexamethasone, | Chen, et al. |
| | | Nicotinamide, 2-phospho-l-ascorbic | 2007 |
| | | acid | |
| 6 | DMEM/F12 | FBS, Insulin, HEPES, EGF, HGF, | Rountree, et |
| | | Dexamethasone, Nicotinamide | al. 2007 |
| 7 | DMEM | FBS | |
| 8 | WEM | FBS, HEPES, Nicotinamide, 2- | Okabe, et al. |
| | | phospho-l-ascorbic acid, NaHCO₃, | 2009 |
| | | Glucose, ITS, EGF, HGF, | |
| | | Dexamethasone | |
| 9 | DMEM | FBS, EGF, bFGF | Oertel, et al. |
| | | | 2008 |
| 10 | *DMEM/F12 | ITS, EGF, E-616452, CHIR99021, | Lv, et al. |
| | | Lysophosphatidic acid, Sphingosine | 2015 |
| | | 1-phosphate, EGF, Nicotinamide, | |
| | | 2-phospho-l-ascorbic acid | |

*Medium supplemented relative to Lv, et al., as shown in Table 2B below
Lazaro et al., Hepatology 38:1095-1106 (2003).
Huch et al., Cell 160:299-312 (2015);
Kubota, et al., PNAS, 97:12132-12137 (2000);
YU, et al., Cell stem cell 13:328-340 (2013);
Chen, et al., Nature protocols 2:1197-1205 (2007).;
Rountree et al., Stem cells 25:2419-2429 (2007);
Okabe, et al., Development 136:1951-1960 (2009);
Oertel, et al., Gastroenterology 134:823-832 (2008);
Lv, et al., Hepatology 61:337-347 (2015).
ITS = insulin-transferrin-sodium selenite supplement;
BSA = bovine serum albumin;
EGF = epidermal growth factor

TABLE 2B
HEM components compared to M10 disclosed in Lv, et al., 2015

| | Components | Lv, et al., 2015 | HEM |
|---|---|---|---|
| | basal medium | DF12 | DF12 and |
| | | | WEM (1:1) |
| | supplement-1 | ITS | B27 without Va |
| | supplement-2 | bovine serum albumin | NONE |
| small | nicotinamide | 5 mM | 5 mM |
| molecule | 2-phospho-L- | 30 lg/mL which is | 200 μM |
| or | ascorbic acid | about 100 μM | |
| growth | EGF | 10 ng/ml | 40 ng/ml |
| factor | CHIR99021 | 3 μM | 3 μM |
| | TGF beta inhibitor | 2 μM E-616452 | 5 μM SB431542 |
| | LPA | 5 μM | 5 μM |
| | S1P | 0.5 μM | 0.5 μM |

HEM can include a suitable antibiotic such as PS

To further generate functional hiHeps from hHPLCs, hHPLCs were cultured until confluent and then treated with 2C medium (HCM with 50 μM forskolin or 50 μM dbcAMP and 10 μM SB431542) for 7-10 days.

### Gene Expression Analysis

Total RNA was isolated by Direct-zol RNA Miniprep (ZYMO RESEARCH) and then reverse-transcribed with TransScript First-Strand cDNA Synthesis SuperMix (TransGen Biotech). RT-qPCR was performed using KAPA SYBR® FAST Universal qPCR Mix (KAPA Biosystems) on a BIO-RAD CFX384™ Real-time System. The quantified values were normalized against the input determined by two housekeeping genes (RPL13A or RRN18S). The RT-qPCR primer sequences are provided in TABLE 3.

TABLE 3
Primers used for RT-qPCR

| Gene | Forward Primer (5′ to 3′) | Reverse Primer (5′ to 3′) |
|---|---|---|
| GAPDH | TGCACCACCAACTGCTTAGC | GGCATGGACTGTGGTCATGAG |
| RPL13A | AGATGGCGGAGGTGCAG | GGCCCAGCAGTACCTGTTTA |
| RRN18S | GTAACCCGTTGAACCCCATT | CCATCCAATCGGTAGTAGCG |
| COL1A1 | CACACGTCTCGGTCATGGTA | AAGAGGAAGGCCAAGTCGAG |
| EPCAM | TGATCCTGACTGCGATGAGAG | CTTGTCTGTTCTTCTGACCCC |
| THY | GGGAGACCTGCAAGACTGTT | CGGAAGACCCCAGTCCA |
| CK8 | GATTGAGGGCCTCAAAGGC | ACAACTTGGCGTTGGCATC |
| CK18 | TGGTCACCACACAGTCTGCTG | TCTCATGGAGTCCAGGTCGAT |
| HNF1B | GCACCTCTCCCAGCATCTCA | GTCGGAGGATCTCTCGTTGC |
| DLK1 | GGGCACAGGAGCATTCATAG | GACGGGGAGCTCTGTGATAG |
| CDH1 | GCCGAGAGCTACACGTTCAC | GTCGAGGGAAAAATAGGCTG |
| MET | GCTGCAAAGCTGTGGTAAACT | CTCCAGCATTTTTACGGACC |
| CLDN3 | CCTGCGTCTGTCCCTTAGAC | CACGCGAGAAGAAGTACACG |
| ALB | GCACAGAATCCTTGGTGAACAG | ATGGAAGGTGAATGTTTCAGCA |
| AFP | CCCGAACTTTCCAAGCCATA | TACATGGGCCACATCCAGG |
| AAT | ACGAGACAGAAGACGGCATT | CCACTGCTTAAATACGGACGA |
| PROX1 | ACAGGGCTCTGAACATGCAC | GGCATTGAAAAACTCCCGTA |
| HNF1A | CCATCCTCAAAGAGCTGGAG | GTGCTGCTGCAGGTAGGACT |
| CEBPA | ACAAGAACAGCAACGAGTACCG | CATTGTCACTGGTCAGCTCCA |
| FOXA3 | GAGATGCCGAAGGGGTATCG | TGATTCTCCCGGTAGTAAGGG |
| CREB3L3 | GCCCTGCCTCTCCTATCATC | ACGGTGAGATTGCATCGTGG |
| CYP1A2 | CTTCGTAAACCAGTGGCAGG | AGGGCTTGTTAATGGCAGTG |
| CYP2A6 | GAGTTCCTGTCACTGTTGCG | GTCCTGGCAGGTGTTTCATC |
| CYP2B6 | CCGGGGATATGGTGTGATCTT | CCGAAGTCCCTCATAGTGGTC |
| CYP2C19 | GAAGAGGAGCATTGAGGACCG | GCCCAGGATGAAAGTGGGAT |
| CYP2C8 | CTCGGGACTTTATGGATTGC | CAGTGCCAACCAAGTTTTCA |
| CYP2C9 | GCCACATGCCCTACACAGATG | TAATGTCACAGGTCACTGCATGG |
| CYP2D6 | GTGTCCAACAGGAGATCGACG | CACCTCATGAATCACGGCAGT |
| CYP3A4 | AGCCTGGTGCTCCTCTATCT | CCCTTATGGTAGGACAAAAT |
| OATP1B1 | TTCAATCATGGACCAAAATCAA | TGAGTGACAGAGCTGCCAAG |
| OAT2 | CACACTCCATCCAGCAAGG | TTGTACCCTACGGTGCTCAG |
| MRP2 | GGGATCTCTTCCACACTGGAT | CATACAGGCCCTGAAGAGGA |
| MRP6 | AAGAACTTGTTTCCCGGCTT | CTCGGTCTCTGGAGCCTTC |
| NTCP | AGGGGGACATGAACCTCAG | AGGTCCCCATCATAGATCCC |
| UGT1A1 | CCATCATGCCCAATATGGTT | CCACAATTCCATGTTCTCCA |
| UGT1A8 | CTGAGACCATTGATCCCAAAG | GGTATCAACTGCCATCAGGG |
| UGT1A10 | CCACAATTCCATGTTCTCCA | TGATGCCCAACATGATCTTC |
| UGT2B15 | GTTTTCTCTGGGGTCGATGA | ATTTGGCTTCTTGCCATCAA |
| UGT2B7 | AACGTAATTGCATCAGCCCT | GGTCATTCTGGGGTATCCAC |
| ASL | CAGTGGACCCCATCATGGAGA | GGCTTTGCTGCCTTGAACATC |
| ASS1 | CTTGGGGCCAAAAAGGTGTTC | GAGGTAGCGGTCCTCATACAG |
| CPS1 | AATGAGGTGGGCTTAAAGCAAG | AGTTCCACTCCACAGTTCAGA |
| CA5A | TAAGGACCCTAACAAGCTGGT | CCGTTTACAGGTGTGCCTCAA |
| ARG1 | GTGGAAACTTGCATGGACAAC | AATCCTGGCACATCGGGAATC |
| OTC | CGGCCCGTGTATTGTCTAGC | TAGCCAGGGTGTCCAAATCTG |
| IFNa2 | CTTGACTTGCAGCTGAGCAC | GCTCACCCATTTCAACCAGT |
| DDX60 | AGTCCAGGATAACAGGATGAATGA | GCTCACGCAAGGAAACACTG |
| DDX3 | CCCTTTGCTGGCTGTACTTC | ATGAGTCATGTGGCAGTGGA |
| IRF7 | AGGGTGACAGGTACGGCTCT | CTCCTGGAGAGGGACAAGAA |
| IRF9 | GCCCTACAAGGTGTATCAGTTG | TGCTGTCGCTTTGATGGTACT |
| PKR | ACTTGGCCAAATCCACCTG | CCCAGATTTGACCTTCCTGA |
| TRIM5 | TCCTTTTTATGACGCCATCC | AGCAGACCCAGTCCCTGAG |
| ZAP | TGTTCAGTCCAGAGAGTTCGTG | GGTGCAACTATTCGCAGTCC |
| BST2 | CTTTTGTCCTTGGGCCTTCT | AGAAGGGCTTTCAGGATGTG |
| ISG15 | TGGACAAATGCGACGAACCTC | TCAGCCGTACCTCGTAGGTG |
| ISG20 | GCTTGCCTTTCAGGAGCTG | ATCACCGATTACAGAACCCG |
| MX1 | GATGATCAAAGGGATGTGGC | AGCTCGGCAACAGACTCTTC |
| MX2 | TGTGCTGTCTCCCTGTCAGA | TGATTTCTCCATCCTGAACG |
| RSAD2 | TGGCTCTCCACCTGAAAAGT | GCCAAAACATCCTTTGTGCT |
| IFIT1 | GCCCTATCTGGTGATGCAGT | GCAGCCAAGTTTTACCGAAG |
| IFITM1 | GCCAACCATCTTCCTGTCC | ATGTCGTCTGGTCCCTGTTC |
| IFITM2 | CCAACCATCTTCCTGTCCC | ATGTCGTCTGGTCCCTGTTC |
| IFITM3 | CCAACCATCTTCCTGTCCC | ATGTCGTCTGGTCCCTGTTC |
| PPARA | AGAGATTTCGCAATCCATCGG | ACTGGTATTCCGTAAAGCCAAAG |
| CAR | TTGCAGAAGTGCTTAGATGCTG | GCCGACAGTATCATGTCTTTCCT |
| RXRA | GACGGAGCTTGTGTCCAAGAT | AGTCAGGGTTAAAGAGGACGAT |
| FXR | CCTGTGAGGGGTGTAAAGGTT | CACTCTTGACACTTTCTTCGCAT |
| PXR | AAGCCCAGTGTCAACGCAG | AGATTTGGGGACCTCCGACTT |

### Immunofluorescence (IF) Staining

Cells were fixed in 4% paraformaldehyde (PFA, DingGuo) at room temperature for 15 minutes and blocked with PBS containing 0.25% Triton X-100 and 5% normal donkey serum (Jackson ImmunoResearch Laboratories, Inc.) at room temperature for 1 hour. Samples were incubated with primary antibodies at 4° C. overnight, washed three times with PBS and then incubated with appropriate secondary antibodies for 1 hour at room temperature in the dark. Nuclei were stained with DAPI (Roche). The primary antibodies used for IF staining are listed in Table 4.

TABLE 4
Antibodies used for immunofluorescence

| Gene | Antibody | Catalog Number |
|---|---|---|
| ALB | Human Albumin Antibody | A80-129A (Bethyl |
| | | Laboratories, Inc.) |
| AFP | Alpha-1-Fetoprotein | ZM-0009 (ZSGB-BIO) |
| CYP3A4 | CYTOCHROME P450 3A4 | AHP622Z (BIO-RAD) |
| CYP1A2 | CYTOCHROME P450 1A2 | AHP610Z (BIO-RAD) |
| CYP2C9 | CYTOCHROME P450 2C9 | AHP617Z (BIO-RAD) |
| CYP2C19 | CYTOCHROME P450 2C19 | AHP618Z (AbD Serotec) |
| CYP2A6 | CYTOCHROME P450 2A6 | AHP612Z (AbD Serotec) |
| CYP2E1 | CYTOCHROME P450 2E1 | AHP621Z (AbD Serotec) |
| HNF1B | Rabbit anti Human HNF1B | SC-22840 (Santa cruz) |
| EPCAM | Mouse anti Human EPCAM | SC-71059 (Santa cruz) |
| CK18 | Mouse anti Human CK18 | ZM-0073 (ZSGB-BIO) |
| UGT1A1 | Human UGT1A1 Affinity | AF6490 (R&D) |
| | Purified Polyclonal Ab | |
| CPR | Mouse anti Human CYPOR | sc-25270 (Santa cruz) |
| CYP2D6 | Rabbit anti Human CYP2D6 | HPA045223 (Sigma) |
| CYP2C8 | Rabbit anti Human CYP2C8 | AHP614Z (AbD Serotec) |
| NTCP | Anti-SLC10A1 antibody | HPA042727 (Sigma) |
| HNF4A | HNF4A antibody | sc-8987 (Santa cruz) |
| E-Cadherin | E-Cadherin (24E10) Rabbit | #3195 (Cell Signaling |
| | mAb | Technology) |
| HBcAg | HBcAg Rabbit Polyclonal | Z2085(ZETA Corporation) |
| | Antibody | |
| HNF1A | HNF1A (H-140) | SC-10791 (Santa cruz) |
| CEBPA | CEBPA Rabbit antibody | 2295s (Cell signaling |
| | | Technology) |
| CEBPB | CEBPB (C-19) | SC-150 (Santa cruz) |

The secondary antibodies used for immunofluorescence were as follows: DyLight® 550 Donkey anti rabbit, DyLight® 550 Donkey anti goat, DyLight® 550 Donkey anti mouse, DyLight® 550 Donkey anti rabbit, DyLight® 650 Donkey anti goat, DyLight® 650 Donkey anti mouse and DyLight® 650 Donkey anti rabbit (all from Abcam). For quantification of ALB-positive cells, images were randomly taken at 10× and 20× magnification at the same exposure using an Operetta High-Content Imaging System (PerkinElmer) and then analyzed by Columbus Image Data Storage and Analysis System.

### Flow Cytometry

Cells were released into single-cell suspension by treating with Accutase (Millipore) at 37° C. for 3-5 minutes. Cells were washed by basal medium. Thoroughly re-suspended cells were added in Fixation/Permeabilization solution (BD, 554714) for 20 min at 4° C. Washed cells two times in 1×BD Perm/Wash buffer. After that cells were conjugated primary antibodies in 200 ul Staining buffer consisting of Perm wash buffer with 2% normal goat serum in 4° C. for 2 hours. Stained cells were washed twice in BD Wash buffer, and incubated in 200 μl Staining buffer containing secondary antibodies. After washed twice in BD Wash buffer, cell were re-suspended in BD Wash buffer and analysis on BD FACSCalibur flow cytometry system. The data were analyzed by FlowJo software.

### Albumin ELISA, α1-Fetoprotein Immunoassay, PAS Staining, LDL Uptake and Oil Red O Staining

Secretion of human albumin was measured using the Human Albumin ELISA Quantitation kit (Bethyl Laboratory) according to the manufacturer's instructions. Secretion human al-fetoprotein was measured using an immunoassay (Roche) by cobas 8000 according to the manufacturer's instructions. The PAS staining system was purchased from Sigma-Aldrich. Cultures were fixed with 4% paraformaldehyde (DingGuo) and stained according to the manufacturer's instructions. For the LDL uptake assay, hiHeps were incubated with 10 μg/ml DiI-Ac-LDL (Invitrogen) for 4 hours and 1 μg/ml Hoechst 33342 (Thermo Fisher Scientific) for 30 minutes at 37° C. and then washed 3 times before imaging using fluorescence microscopy. Lipid detection was performed with a Lipid (Oil Red 0) Staining Kit (Sigma) according to the manufacturer's instructions.

### Measurements of Cytochrome P450 Activity

The methods for measuring the CYP450 activity were described previously. Briefly, hiHeps and HepG2 cells were dissociated and suspended to measure their CYP450 activity, and the CYP450 activity in F-PHHs was measured immediately after isolation. Commercial cryopreserved PHHs were purchased from RILD (Shanghai) and used immediately after resuscitation. One 500 μl reaction contained 2.5×10⁵ cells and the indicated substrates. After incubation for 15-30 minutes at 37° C. in an orbital shaker, the reactions were stopped by the addition of sample aliquots to tubes containing triple the volume of quenching solvent (methanol) and were frozen at −80° C. Isotope-labeled reference metabolites were used as internal standards for further mass spectrometry (ultra-performance liquid chromatography-tandem mass spectrometry, UPLC/MS/MS) analysis. The details of the substrates, internal standards and other relevant information are listed in Table 5.

TABLE 5
Drugs used for measurement of CYP450 activity

| | Fraction of | Fraction of | | | | |
|---|---|---|---|---|---|---|
| | metabolizing | metabolizing | | | | |
| CYP450s | marketed | marketed | Standard | Concentration | | |
| isoforms | drugs* | drugs^{#} | substrate | of substrate | Internal standard | Product for detection |
| CYP3A4 | 30.2% | 50.0% | Testosterone | 200 μM | 6β- | 6β- |
| | | | | | Hydroxytestosterone- | Hydroxytestosterone |
| | | | | | [D7] | |
| CYP2D6 | 20.0% | 30.0% | Dextromethorphan | 15 μM | Dextrorphan-[D3] | Dextrorphan |
| CYP2C9 | 12.8% | 10.0% | Difclofenac | 25 μM | 4′-Hydroxydiclofenac- | 4′-Hydroxydiclofenac |
| | | | | | [13C6] | |
| CYP2B6 | 7.2% | N.A. | Bupropion | 500 μM | Hydroxybupropion- | Hydroxybupropion |
| | | | | | [D6] | |
| CYP1A2 | 8.9% | 4.0% | Phenacetin | 100 μM | Acetomidophenol- | Acetaminophen |
| | | | | | [13C2, 15N] | |
| CYP2C19 | 6.8% | 2.0% | S-mephenytoin | 250 μM | 4′-Hydroxymephenytoin- | 4′-Hydroxymephenytoin |
| | | | | | [D3] | |
| CYP2C8 | 4.7% | N.A. | Paclitaxel | 20 μM | 6α-hydroxypaclitaxel- | 6α-hydroxypaclitaxel |
| | | | | | [D5] | |

*Zanger, et al., Pharmacology & therapeutics 138, 103-141 (2013).
^{#}Zhou, et al. Drug metabolism reviews 41, 89-295 (2009).

UPLC/MS/MS analyses were performed using an ACQUITY H-Class UPLC System (Waters) coupled to a Sciex API4500Q-trap Mass Spectrometer (SCIEX). The analytical column was an ACQUITY UPLC® BEH C18 1.7 μm 2.1*50 mm coupled with a preguard column. The results are expressed as picomoles of metabolite formed per minute and per million cells. hiHeps were used directly to measure the activity of CYP3A4, CYP2B6, CYP2C8 and CYP2D6. To measure the activity of CYP1A2, hHPLCs were cultured in 2C medium with additional 3 μM CHIR and 2 μM U0126. To measure the activities of CYP2C9 and CYP2C19, hiHeps were cultured with 10 μM rifampin.

To measure the induced activity of CYP3A4, CYP2B6 and CYP1A2, hiHeps were further cultured in HCM with 50 μM rifampin, 1 mM phenobarbital, 50 μM β-naphthoflavone or 10 μM lansoprazole for 3 days. Vehicle-treated groups were used to determine the basal activity. To measure the induced activity of PHHs, PHHs were cultured and induced under sandwich methods.

### Measurements of Hepatic Clearance

Measurements of clearance were performed as previously described (McGinnity, et al. Drug metabolism and Disposition: The Biological Fate of Chemicals 32, 1247-1253 (2004); Obach, et al. Drug metabolism and Disposition: The Biological Fate of Chemicals 36, 1385-1405 (2008)). Briefly, a 1×10⁶ cells/ml cell suspension and 2× drug solutions were prepared in incubation medium (William's E medium, 10 mM HEPES [pH 7.4], and 1% GlutaMAX). Reactions were started by adding 500 μl of drug solutions to 500 ul of hiHeps, giving a final substrate concentration of between 1 and 2 μM. The detailed information of the substrates is listed in Table 6.

TABLE 6
Drugs used for measurement of hepatic clearance

| | Responsible | | |
|---|---|---|---|
| | enzymes for | Concentration | |
| Drugs | metabolism | (μM) | Internal standards |
| Midazolam | CYP3A4 | 1 | Hydroxymidazolam-[13C3] |
| Verapamil | CYP3A4 | 1 | Verapamil-hydrochloride |
| Diclofenac | CYP2C9 | 1 | 4'-Hydroxydiclofenac-[13C6] |
| Phenacetin | CYP1A2 | 1 | Acetomidophenol-[13C2, 15N] |
| Naloxone | UTG2B7 | 1 | Naloxone-[D5] |

These concentrations were selected to be below the Km for most substrates but to still have sufficient analytical sensitivity. Reactions were performed in an orbital shaker in an incubator at 37° C. Then, 80 μl aliquots were removed at 0, 15, 30, 60, 90, 120, 180 and 240 minutes, and samples were quenched in 240 μl of methanol containing an isotope-labeled reference and frozen at −80° C. The substrates were quantified using the validated traditional LC-MS/MS methods described above. Assays were performed in triplicate.

In vitro intrinsic clearance (CLint) was determined using the rate of parent disappearance as described previously (Houston, Biochemical pharmacology 47, 1469-1479 (1994); Levy et al., Nature biotechnology 33, 1264-1271 (2015)). The slope (−k) of the linear regression from the log [substrate] versus time plot was determined. Because the elimination rate constant was k=0.693/t1/2, an equation expressing CLint in terms of t1/2 of parent loss was derived as follows: Clint=volume×0.693/t1/2. The hepatocyte CLint (units, μl/min/10⁶ cells) was scaled to the in vivo CLint (units, ml/min/kg) using the following physiological parameters: human liver weight of 22 g/kg body weight and hepatocellularity of 120×106 cells/g of liver. The projection of human in vivo hepatic clearance (CLₕ) was made using an adapted version of the nonrestrictive well stirred model as follows: CLₕ=(CLint×Qₕ)/(CLint×Qₕ), where Qₕ is hepatic blood flow (human Qₕ=20 ml/min/kg). No correction factor was made for any differential in vitro and in vivo binding, and the distribution of the drug between the plasma and blood was assumed to be unity.

### Toxicity Assay

For the toxicity assay, hiHeps, HepG2 cells and PHHs were cultured in 96-well or 384-well plates. Compounds with 7-8 concentration dilutions were prepared in DMEM with 3% FBS to test HepG2 cells or in HCM to test PHHs and hiHeps. The final DMSO concentration under all conditions was consistent. The detailed information of the tested compounds is listed in Table 7 ((Seglen, et. Al., Methods in Cell Biology 13, 29-83 (1976)), Zhong et. Al., Clin Chim Acta. 412, 1905-1911 (2011))).

TABLE 7
Compounds used for toxicity prediction

| | The highest | | |
|---|---|---|---|
| | tested | | |
| | concentration | | |
| Compound | (mM) | Label* | Toxicity mechanism |
| Acetaminophen | 3 | Representative drug | Bioactivation; Mitochondrial |
| | | | dysfunction |
| Aflatoxin B1 | 1 | Representative drug | DNA damage, Bioactivation |
| Maraviroc | 3 | DILI concern by FDA | Immunoreaction; Bioactivation |
| Carbamazepine | 3 | DILI concern by FDA | Bioactivation; Mitochondrial |
| | | | dysfunction |
| Bosentan | 3 | DILI concern by FDA | Direct toxicity; Bioactivation |
| Etodolac | 3 | DILI concern by FDA | Idiosyncratic toxicity |
| Fenoprofen | 3 | DILI concern by FDA | Idiosyncratic toxicity |
| Flutamide | 3 | DILI concern by FDA | Bioactivation |
| Cyclosporine A | 3 | DILI concern by FDA | Reactive oxygen species |
| Trazodone | 1.5 | DILI concern by FDA | Reactive oxygen species; |
| | | | Bioactivation |
| Leflunomide | 3 | DILI concern by FDA | Bioactivation |
| Rifampin | 1.5 | DILI concern by FDA | Reactive oxygen species |
| Tolcapone | 3 | DILI concern by FDA | Bioactivation; Mitochondrial |
| | | | dysfunction |
| Diclofenac | 3 | DILI concern by FDA | Bioactivation; Mitochondrial |
| | | | dysfunction |
| Tacrine | 3 | DILI concern by FDA | Reactive oxygen species |
| Ticlopidine | 3 | DILI concern by FDA | Idiosyncratic toxicity |
| Docetaxel | 3 | DILI concern by FDA | Mitochondrial dysfunction |
| Zafirlukast | 3 | DILI concern by FDA | Idiosyncratic toxicity |
| Simvastatin | 3 | DILI concern by FDA | Mitochondrial dysfunction |
| Nortriptyline | 3 | DILI concern by FDA | Idiosyncratic toxicity |
| Tipranavir | 3 | DILI concern by FDA | Bioactivation |
| Nefazodone | 1 | DILI concern by FDA | Bioactivation; Mitochondrial |
| | | | dysfunction |
| Chlorpromazine | 3 | DILI concern by FDA | Bioactivation; Idiosyncratic |
| | | | toxicity |
| Amiodarone | 0.3 | DILI concern by FDA | Phospholipidosis; Steatotic; |
| | | | Mitochondrial dysfunction |
| Sunitinib | 1.5 | DILI concern by FDA | Hyperammonemia |

*Chen et al., Drug Discovery Today 16, 697-703 (2011); Levy et al., Nature Biotechnology 33, 1264-1271 (2015).

Compounds were tested in bipartite in a dilution series by half-log concentration increments. After the cells were treated with compounds for 24 hours, the supernatant was discarded and modified WEM (William's E medium supplemented with 2% B27 and 1% GlutaMAX) containing fluorescent probes was added to cells. The following three fluorescent probes were simultaneously used to monitor cells in culture: 2 μM CellTrace™ Calcein Red-Orange AM (Thermo Fisher Scientific), 0.1 μM MitoTracker™ Deep Red FM (Thermo Fisher Scientific) and 1 μg/ml Hoechst 33342 (Thermo Fisher Scientific). After incubation for 30 minutes, the supernatant was discarded and cells were washed twice. Images were acquired using the Operetta High-Content Imaging System (PerkinElmer) with a 10× objective. The supernatant containing fluorescent probes was discarded, and cells were further incubated with modified WEM containing 9.1% CCK-8 (Dojindo). After 1-4 hour chromogenic reaction in the incubator, the absorbance at 450 nm was read using a SpectraMax i3× (Molecular Devices). Image data were analyzed online using the Columbus Image Data Storage and Analysis System (PerkinElmer) with the following steps: (1) identification and counting of nuclei; (2) identification and counting of live cells labeled by CellTrace™ Calcein Red-Orange AM; and (3) identification and counting of live cells labeled by MitoTracker™ Deep Red FM. The viability of each parameter transformed from image data and the CCK-8 assay data were entered into Excel. The cellular viability of each parameter was expressed as the live cell ratio and normalized to the negative control. The TC50 values of these four parameters were calculated separately, and the minimum TC50 value was used as the final TC50.

### Steatosis and Phospholipidosis Assay

Imaging and quantification of intracellular lipids were performed using HCS LipidTOX™ Deep Red Neutral Lipid Stain (1000×) (Thermo Fisher Scientific) according to the manufacturer's directions. Briefly, hiHeps were incubated with compounds with the following concentration gradient: 100%, 80%, 60%, 40%, 20% and 0% TC50. The TC50 vales of amiodarone, tetracycline hydrochloride and rifampin were 30 μM, 400 μM and 100 μM, respectively, which were all rounded for ease of use. The final DMSO concentration of all of the tested wells and control wells was 0.1%. After incubation with compounds for 24 hours, cells were fixed with 4% PFA. Nuclei were stained with DAPI, and lipids were stained with 1× Neutral Lipid Stain. Images were captured using an Operetta High-Content Imaging System (PerkinElmer) and analyzed with Columbus Image Data Storage and Analysis System.

Quantification of intracellular phospholipids was performed using HCS LipidTOX™ Red Phospholipidosis Detection Reagent (1000×) (Thermo Fisher Scientific) according to the manufacturer's instructions. Briefly, hiHeps were cultured at a final concentration of 1× Phospholipidosis Detection Reagent and the following tested compounds: amiodarone (TC50≈30 μM), chlorpromazine (TC50≈25 μM) and rifampin (TC50≈100 μM). The TC50 values were all rounded for ease of use. Compounds were tested in the following concentration gradient: 80%, 60%, 40%, 20% and 0% TC50. The final DMSO concentration of all of the tested wells was consistent. After incubation with the compounds and detection reagent for 24 hours, cells were fixed with 4% PFA, and nuclei were stained with DAPI. Images were captured using an Operetta High-Content Imaging System and analyzed with Columbus Image Data Storage and Analysis System.

### Drug-Drug Interaction Assay

hiHeps in the DMSO group were cultured in HCM supplemented with DMSO for 3 days. hiHeps in the RIF group were cultured in HCM supplemented with 20 μM rifampin for 3 days. hiHeps in the RIF+KC group were cultured in HCM supplemented with 20 μM rifampin for 3 days, and KC was added on the third day. The final DMSO concentration of these conditions on each day was unified to the highest concentration among these three groups. Aflatoxin B1 and flutamide were further tested on these cells following the toxicity assay.

### HBV Infection and Analysis of HBV Replication Intermediates

HBV was concentrated from the supernatant of the HBV-producing HepAD38 cell line using centrifugal filter devices (Centricon Plus-70, Biomax 100.000, Millipore Corp., Bedford, Mass.) and titered by HBV-DNA RT-qPCR (KHB, Shanghai, China). hiHeps and PHHs were infected with HepAD38-derived HBV at a multiplicity of infection (MOI) around 300 in HCM containing 2% DMSO and 4% PEG 8000 (Sigma Aldrich) for 16-20 hours. After infection, cells were washed 9 times with PBS and cultured in advanced 2C medium. HepG2-NTCP cells were infected at the same MOI in DMEM containing 2% FBS, 2% DMSO and 4% PEG 8000, and they were cultured in DMEM supplemented with 2% FBS and 2% DMSO. hHPLCs were infected in HEM containing 4% PEG 8000. The medium was changed every 3 days, and the supernatants were collected. For inhibition of the HBV life cycle, lamivudine (LAM) (TargetMol) and entecavir (ETV) (TargetMol) were used at 1 μM and 0.5 μM, respectively, during and after infection. The viral entry inhibitor N-terminal myristoylated peptides (MYR, (provided by Hui Zhuang's lab)) were used at 500 μM during infection, IFN-α (Genway) was used at 1000 U/mL after infection. The HBV viral antigens HBsAg and HBeAg were examined using 50 μl of supernatants with commercial ELISA kits (Autobio, Henan, China) following the manufacturer's instructions. Extracellular HBV DNA quantification was performed via DNA extraction using a HBV detection kit (KHB, Shanghai, China). Intracellular HBV-DNA was extracted using a DNeasy Blood & Tissue kit (QIAGEN) and quantified using the following specific primers by real-time PCR: 5′-GAGTGTGGATTCGCACTCC-3′ (forward) and 5′-GAGGCGAGGGAGTTCTTCT-3′ (backward). The viral genome equivalent copies were calculated based on a standard curve generated from samples with known copy numbers. Real-time PCR was performed using KAPA SYBR® FAST Universal qPCR Mix (KAPA Biosystems) and a BIO-RAD CFX384™ Real-time System.

To quantify HBV-specific RNAs, total RNA was isolated from HBV-infected cells using the Direct-zol RNA Miniprep kit (ZYMO RESEARCH). Quantification of HBV-specific RNAs was performed as previously described. Approximately 400 ng of total RNA was reverse transcribed into cDNA with TransScript First-Strand cDNA Synthesis SuperMix (TransGen Biotech). The following primers were used for the HBV 3.5-kb transcripts: 5′-GAGTGTGGATTCGCACTCC-3′ and 5-GAGGCGAGGGAGTTCTTCT-3′. The following primers were used for the total HBV-specific transcripts: 5′-TCACCAGCACCATGCAAC-3′ and 5′-AAGCCACCCAAGGCACAG-3′. Quantification of these transcripts was performed by real-time PCR.
HBV cccDNA was quantified using rolling circle amplification in combination with real-time PCR as previously described by Yanwei Zhong et al. Single-stranded and relaxed circular DNAs were degraded before amplification by treating the DNA templates with plasmid-safe adenosine triphosphate (ATP)-dependent deoxyribonuclease DNase (PSAD, Epicentre Technologies). PSAD-treated samples were subjected to rolling circle amplification (RCA) prior to real-time PCR mediated by cccDNA-selective primers. Four pairs of primers were designed for mediating RCA:

RCA1
AATCCTCACAATA *C*C 99-113
RCA2
ACCTATTCTCCTC *C*C 1758-1744
RCA3
CCTATGGGAGTGG *G*C 510-524
RCA4
CCTTTGTCCAAGG *G*C 2689-2675
RCA5
ATGCAACTTTTTC *A*C 1686-1700
RCA6
CTAGCAGAGCTTG *G*T 29-15
RCA7
TAGAAGAAGAACT *C*C 2240-2254
RCA8
GGGCCCACATATT *G*T 2599-2585

The reaction was performed with Phi29 DNA polymerase (New England Biolabs, Worcester, Mass.) at 30° C. for 16 hours and terminated at 65° C. for 10 minutes. Using the RCA products as a template, HBV cccDNA was further amplified and quantified with real-time PCR mediated by a pair of cccDNA-selective primers (5′-GGGGCGCACCTCTCTTTA-3′ 1521-1538; 5′-AGGCACAGCTTGGAGGC-3′ 1886-1870).

For southern blot analysis of HBV cccDNA, the method described by Cal et al., (Methods in Molecular Biology 1030, 151-161 (2013)) with modifications, was used. Briefly, a modified Hirt method was used to extract proteins-free viral DNA as described, and half of the extracted DNA sample was treated with Spe1 (NEB). For southern blotting, the DNA was separated on a 1.2% agarose gel and then transferred to a Hybond-XL membrane. A 3.2 kb and 2.0 kb HBV DNA fragments was also run on the same agarose gel to serve as the molecular marker. Southern blot was performed with the DIG High Prime DNA Labeling and Detection Starter Kit II (Roche, 11 585 614 910), with reference to “Roche Techniques for Hybridization of DIG-labeled Probes to a Blot”. Lane 1-4 were Hirt DNA from hiHeps infected with HBV from patient sera, and lane 5-6 were Hirt DNA from hiHeps infected with HepAD38-derived HBV.

### RNA Sequencing and Bioinformatics Analysis

Total RNA was isolated from HEFs, hHPLCs, hiHeps, fetal liver cells, HepG2 cells, and F-PHHs using the RNeasyMini kit (QIAGEN). RNA sequencing libraries were prepared using the NEBNext Ultra™ RNA Library Prep kit for Illumina (NEB, USA) following the manufacturer's recommendations. The fragmented and randomly primed 150-bp paired-end libraries were sequenced on Illumina Hiseq 4000 platform. The generated sequencing reads were mapped against the human genome build hg19 using STAR, and the read counts for each gene were calculated using featureCounts.

Gene expression was normalized by DESeq2, and low expression genes with total counts across all samples less than 1 were excluded. Unsupervised hierarchical clustering of RNA-seq data was conducted by the hclust package in R (R 3.4.3, https://www.r-project.org). Heatmaps were generated by the pheatmap package.

MicroRNA Sequencing and qPCR Analysis

For microRNA sequencing, total RNA was extracted using TRNzol Universal (TIANGEN). RNA sequencing libraries were prepared using the NEB Next® Multiplex Small RNA Library Prep Set for Illumina (NEB, USA) following the manufacturer's recommendations. The fragmented and randomly primed 140-160 bp single-end libraries were sequenced on Illumina Hiseq 2500 platform. First, the quality of raw microRNA deep sequencing data was checked by software FastQC. Subsequent clean fasta format sequencing reads generated from the sequencer mapped against the human genome build hg19 and calculated read counts by miRDeep2. All of the identified miRNAs were compared with those represented in release 22 of miRBase (March 2018) (Kubota, et al., PNAS, 97, 12132-12137 (2000)). And then microRNA expression was normalized by DESeq2. Heatmaps were performed by the pheatmap package (pheatmap 1.0.8) in R (R 3.4.3). Key hepatic microRNAs were selected according to previous reports (Szabo, et al., Nature Reviews. Gastroenterology & hepatology 10, 542-552 (2013); Willeit, et al., European Heart Journal 37, 3260-3266 (2016); Lazaro et al., Hepatology 38, 1095-1106 (2003))

For qPCR analysis of microRNA, total RNA was extracted using TRNzol Universal (TIANGEN) and then reversely transcribed into cDNA using miRcute miRNA First-strand cDNA (TIANGEN). qPCR was performed using miRcute miRNA qPCR Detection (TIANGEN) with primers listed below. The relative expression of microRNAs are normalized to U6 spliceosomal RNA.

miR-15a
TAGCAGCACATAATGGTTTGTG
miR-378
TCCTGACTCCAGGTCCTGTGT
miR-30e
TGTAAACATCCTTGACTGGAAG
miR-192-3p
CTGCCAATTCCATAGGTCACAG
miR-122
TGGAGTGTGACAATGGTGTTTG
miR-194
TGTAACAGCAACTCCATGTGGA
miR-25
CATTGCACTTGTCTCGGTCTGA
miR-26b
TTCAAGTAATTCAGGATAGGT
U6-forward
CTCGCTTCGGCAGCACA
U6-reverse
AACGCTTCACGAATTTGCGT

### DNA Methylome Analysis

Cells were released into cell suspension and washed by PBS. DNA were extracted using QIAamp DNA Mini Kit (Qiagen). DNA methylation level was measured by Illumina 850K Genechip using the Illumina Infinium HD Methylation Assay (Illumina) according to the manufacturer's instructions. The methylation level raw data was performed by illumine 850k platform. The CpG methylation level for all sites and promoter regions were calculated by RnBeads (RnBeads_1.12.1, 1, https://github.com/thomasvangurp/epiGBS/tree/master/RnBeads) R package. Unsupervised hierarchical clustering of all DNA methylation sites and promoters were performed by hclust package in R (R 3.4.3). Heatmaps were generated by pheatmap package. The visualization of particular regions of all samples were done by software IGV (IGV_2.4.14, 4, https://github.com/igvteam/igv).

### Growth Curve and Doubling Times

The cell numbers of fibroblasts and hHPLCs at different passages were counted at days 0, 2, 3 and 4 after plating cells in 12-well plates. Given the measurements of the growing quantity, q1 (units, cell) at day 0 and q2 at time t2 (units, hour), the doubling time, Td (units, hour), was calculated as follows:

Td=(t2× log 2)/(log(q2/q1)).

### Analysis of RNA-Seq Data for the Activation of Silent Hepatic Genes Marked by H3K9Me3 in Fibroblasts

The referenced RNA-seq raw data analyzed in FIG. 6B were downloaded from GEO (Accession ID: GSE103078). The raw reads were mapped to hg19 by tophat2, and HTseq was used to calculate the gene expression levels. The expression data from GSE103078 and data from our study were normalized by the quantile method using the R package (preprocessCore). The gene activation levels were calculated using the silent hepatic genes, which were marked by H3K9me3 in fibroblasts and defined by Kenneth S. Zaret (Becker et al., Molecular Cell 68, 1023-1037 e1015 (2017)) Using the method described, log 2 gene expression for hiHeps was calculated on a relative scale, with 0% representing the log 2 gene expression of fibroblasts and 100% representing the log 2 gene expression of primary human hepatocytes. Negative values (the gene expression of hiHeps was lower than that of fibroblasts) were rounded to 0%. The distributions of the relative expression values from GSE103078 and data from our study were visualized by violin plots using ggplot2 package in R.

### Statistical Analysis

The sample size was not predetermined by any statistical method and depended on the experiment type based on the standard practice in the field of lineage reprogramming and stem cell biology as well as on the basis of preliminary data. The experiments were not randomized, and the investigators were not blinded to the allocation during experiments and outcome assessment. For all measurements, ‘n’ represents the number of biological replicates. Experiments were independently replicated at least twice, and representative data are shown. P values for the purpose of group comparisons were calculated using one-way ANOVA. Correlations were evaluated using Pearson correlation coefficients. The level of significance in all graphs is represented as follows: *P<0.05, **P<0.01, ***P<0.001. Unless described otherwise, standard statistical analyses were performed with GraphPad Prism 7 using default parameters. All of the error bars represent SEM.

### Code and Data Availability

The bioinformatics scripts used to analyze the data presented in the study is available on GitHub. The RNA sequencing data is available in the Gene Expression Omnibus (GEO) under the accession number GSE112330. All figures have associated raw data, and data supporting the conclusions of this study are available from the corresponding author upon reasonable request.

### Results and Discussion

The present studies show that that a 5-TF cocktail, containing HHEX, HNF6A, GATA4, HNF4A and FOXA2 led to the production of albumin (ALB)- and alpha-fetoprotein (AFP)-positive cells, suggesting hepatic fate conversion from HEFs (data not shown).

To capture and expand cells with the property of hepatic progenitors, 5-TFs-overexpressing HEFs were with 10 different media (M1-10; TABLE 2A) reported for culturing HPCs, and M10 gave the highest yield of 2.7% ALB+ cells (FIG. 1B). After the further supplementation of M10 (TABLE 2B), hepatic expansion medium (HEM) was obtained, which promoted the generation and expansion of epithelial colonies, and ALB+ cells robustly accounted for approximately 75% of all cells at 40 dpi (data not shown and FIG. 1B-D). During reprogramming, HPC markers ALB, AFP and EpCAM were greatly upregulated and fibroblast markers COL1A1 and THY1 were downregulated (FIG. 1F). The 5-TF and HEM, combination establishes a robust system to generate proliferative hepatic cells from fibroblasts.

Key hHPC markers were significantly expressed in reprogrammed cells (data not shown and FIG. 1E). Global transcriptome profiling revealed that the reprogrammed cells were close to hFLCs but distinct from HEFs and freshly isolated primary human hepatocytes (F-PHHs) (FIG. 1G; and data not shown). Additionally, hHPC-enriched genes, including those with known roles for hHPCs, were greatly upregulated (for example AFP, ALB, CDH1, DLK1, EPCAM, HES1, HNF1B, KRT18, KRT8, MET, PROX1, TTR (data not shown). Collectively, these results indicated that these reprogrammed cells acquired an hHPC identity and they were termed as human hepatic progenitor-like cells (hHPLCs).

Next, the hHPC-like cells (hHPLCs) were induced to further differentiate into functional hepatocytes, designated as human induced hepatocytes (hiHeps). First, additional studies identified that a combination of cAMP activator and TGFβ inhibitor, could maintain essential functions of PHHs (FIG. 2A). Notably, when hHPLCs were cultured in this medium for 10 days, they formed differentiated cells exhibiting the typical hepatocyte-morphology similar to cultured PHHs and were immunopositive for E-cadherin and hepatic-TFs HNF4A, HNF1A, CEBPA, CEBPB (data not shown). By flow cytometry analysis, the ALB+ cells counted for over 90% of hiHeps (FIG. 2C). Second, the expression levels of major mature hepatocyte-functional genes were found dramatic upregulated in hiHeps compared with hHPLCs, and were comparable to those in F-PHHs and adult liver tissue (AL) (FIGS. 2D and 2B). Third, the expression of functional genes ALB and CYP450s, were stably maintained for at least 35 days, during which fetal marker AFP was abolished (data not shown; FIGS. 2E and 2F). Additionally, other fetal hepatocyte markers including DLK1 and EPCAM were also downregulated in hiHeps (data not shown). Fourth, hiHeps were immunopositive for key drug-metabolizing enzymes of CYP450s, UGT1A1 and POR (data not shown). Fifth, hiHeps were competent for low-density lipoprotein (LDL) uptake, fatty droplets synthesis and glycogen synthesis (data not shown). Lastly, ALB secretion of hiHeps was maintained for at least 35 days at a comparable level to that of PHHs (FIGS. 2G and 2H). These data indicated that hHPLCs gave rise to functional hepatocytes.

In global gene expression analysis, hierarchical clustering revealed that hiHeps were clustered closely with F-PHHs and ALs (FIG. 2I). Importantly, key hepatic TFs and genes involved in hepatic metabolism were upregulated, meanwhile fibroblast signature gene expression were undetectable in hiHeps (data not shown). On global DNA methylome level, hiHeps was closely clustered with F-PHHs and was separated from HEFs (data not shown, FIG. 3E). The methylation changes in specific gene regions also occurred (data not shown). Additionally, microRNA profiling showed that hiHeps clustered closely with F-PHHs (data not shown), and the expression of major hepatocyte-associated microRNAs, including miR122, in hiHeps were comparable to F-PHHs (FIG. 3F). Overall, the activation of hepatocyte gene regulatory networks and the DNA methylation pattern clearly reflected the establishment of hepatocyte-identity in hiHeps.

To exclude donor variability of the reprogramming process, additional four hiHeps cell lines reprogrammed from fibroblasts of three different donors and one commercialized human fibroblast line HFF-CRL-2097 were established. All hiHeps showed similar global gene expression profile (FIGS. 3G and 3H). CRL-2097-derived hiHeps acquired functional phenotypes indicated by typical hepatocyte morphology, hepatic gene expression and hepatocyte function analysis (data not shown; FIG. 3I; 4A-4B).

hiHeps could functionally replace PHHs in several in vitro applications including drug metabolism, toxicity prediction, liver disease modeling. For drug metabolism, the function of CYP450s and seven major drug-metabolizing CYP450s in hiHeps was determined by mass spectrometry (FIG. 3A). Importantly, the metabolic activities of these CYP450s in hiHeps were all comparable to those in PHHs (FIG. 3A). Furthermore, hiHeps also showed the potential of the drug clearance prediction, for the scaled in vivo hepatic clearance (CLₕ) of hiHeps was comparable to the observed in vivo CLₕ in previous reports (Table 8) (Lilja et al., Transplantation 64:1240-1248 (1997)).

TABLE 8
Scaled in vivo hepatic clearance (CLₕ) of hiHeps was compared to the observed in vivo CLₕ

| | | | Scaled in | | |
|---|---|---|---|---|---|
| | | In vitro Clint | vivo CLₕ | | |
| | Responsible | hiHeps by | by hiHeps | Observed CLₕ | Prediction |
| Drugs | Enz | (μL/min/10⁶) | (ml/mim/kg) | in vivo | Accuracy |
| Midazolam | CYP3A4 | 2.8 | 5.3 | 5.3 | Within 2 fold |
| Verapamil | CYP3A4 | 7.8 | 10.1 | 18 | Within 2 fold |
| Diclofenac | CYP2C9 | 4.5 | 7.4 | 7.6 | Within 2 fold |
| Phenacetin | CYP1A2 | 18.3 | 14.1 | 21 | Within 2 fold |
| Naloxone | UGT2B7 | 14.2 | 13 | 18 | Within 2 fold |

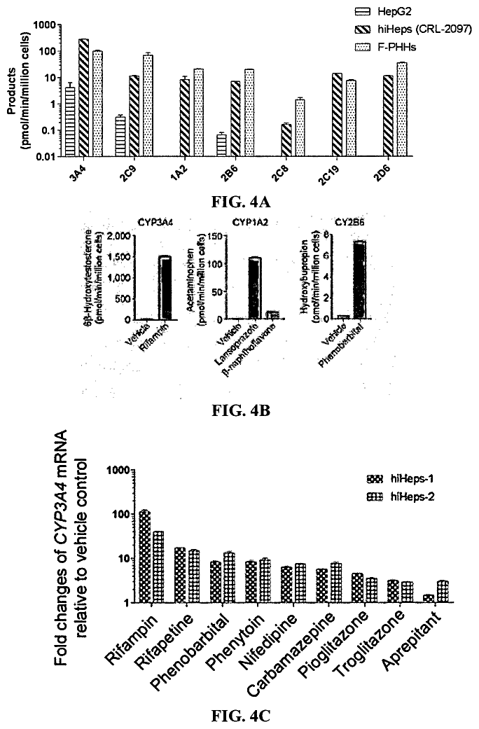

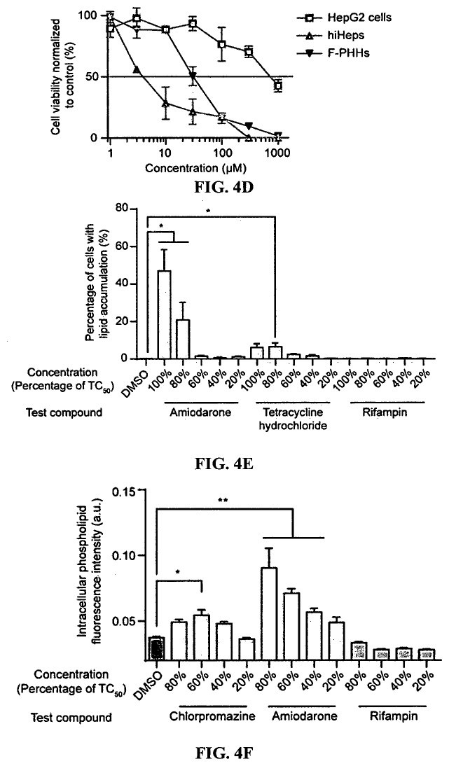

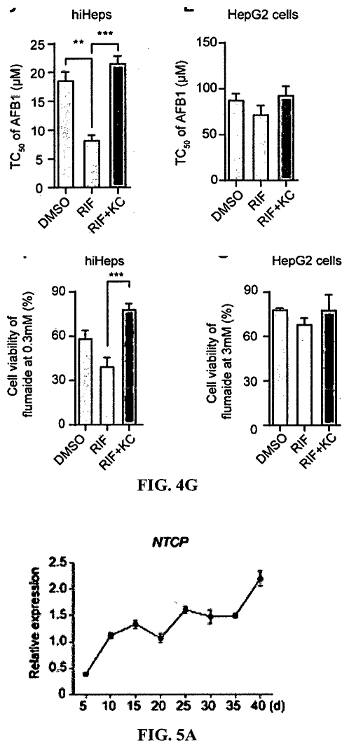

Next, the data shows that hiHeps modulate the activity of CYP450s through nuclear receptor activation. When hiHeps were exposed to PXR agonist (rifampin), AhR agonists (β-naphthoflavone and lansoprazole) and CAR agonist (Phenobarbital) to induce CYP3A4, CYP1A2 and CYP2B6, respectively, these CYP450s could be induced by their corresponding inducers, all comparable to the results in PHHs (FIG. 3B). hiHeps also responded to structurally different CYP3A4 inducers (FIG. 4C). These data suggested equivalent innate CYP450-metabolic activities of hiHeps to PHHs, and the suitability of hiHeps for in vitro drug-metabolism studies. The data also showed that hiHeps provided an excellent in vitro system for predicting liver drug toxicity as PHHs. First, 25 hepatotoxins were tested on hiHeps (Table 7) (Seglen, et. Al., Methods in Cell Biology 13, 29-83 (1976)). Compound toxicity was characterized by TC50, the concentration causing 50% reduction of cell viability (FIG. 4D). Notably, TC50 profiles of hiHeps for these compounds were not discriminated from those of PHHs (FIG. 3C). Interestingly, bioactivation compounds showed lower TC50 profiles in hiHeps and PHHs than in HepG2 cells, consistent with the robust drug-metabolizing activity of hiHeps (FIG. 3C). A chronic hepatotoxin, troglitazone, caused extensive cell death after prolonged 9 days of drug exposure with a non-lethal concentration in line with previous PHH studies (FIG. 3D) (Hou et al., Science 341, 651-654 (2013)). Second, drug-induced pathological effects could be recapitulated with hiHeps. Severe and dose-dependent steatosis and phospholipidosis were detected in hiHeps upon exposure to pathology-causing hepatotoxins (data not shown and FIGS. 4E and 4F). Lastly, hiHeps could evaluate toxicity caused by drug-drug interactions (DDIs). The toxicity of two bioactivation drugs, aflatoxin B1 (AFB1) and flutamide, increased after induction with rifampin in hiHeps, and hiHeps were further rescued by CYP3A4 inhibitor ketoconazole (FIG. 4G).

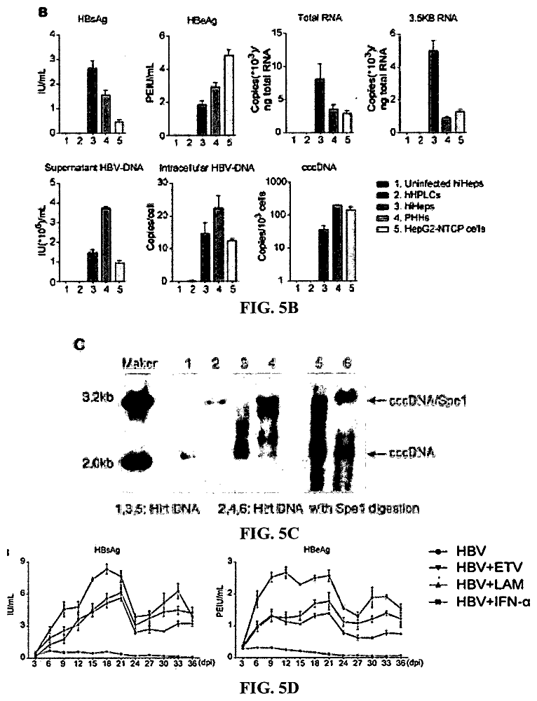

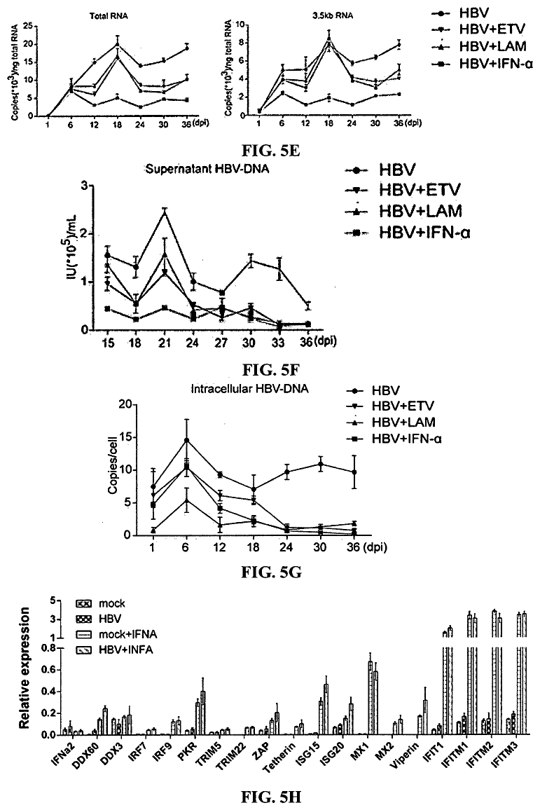

The studies next tested whether hiHeps could serve as an in vitro model to recapitulate HBV infection. First, the expression of HBV receptor NTCP in hiHeps was confirmed (FIGS. 2D and 5A). Notably, HBV-infected hiHeps were immunopositive for HBcAg (data not shown). The secretion of HBsAg and HBeAg, and the expression of HBV-DNA, -RNA, -cccDNA in hiHeps was also analyzed. These HBV markers in hiHeps were comparable to those in PHHs and HepG2-NTCP cells (FIG. 5B). Importantly, the presence of cccDNA was confirmed by southern blot (FIG. 5C). Dynamic analysis revealed that the secretion of HBsAg and HBeAg gradually increased and peaked at 20 dpi, which was correlated with the kinetics of HBV-RNA expression (FIG. 5D-E). The supernatant and intracellular HBV-DNA retained their expression for 36 days (FIG. 5F-G). These results collectively indicated that hiHeps are robustly permissive for long-term HBV infection in vitro.

Subsequent studies evaluated the response of hiHeps to anti-HBV compounds. A viral entry inhibitor N-terminal myristoylated peptides (MYR) showed a significant inhibition on HBV proteins consistent with a low expression of HBV-RNA (FIG. 5I). The nucleoside analogs entecavir (ETV) and lamivudine (LAM) greatly inhibited HBV-DNA, especially during prolonged treatment (FIG. 5D-G). Moreover, interferon-alpha (IFN-α) showed inhibitory effect on all major HBV markers stated above, which was associated with an upregulation of many IFN-stimulated genes especially antiviral effectors (FIG. 5H). This suggested an innate antiviral immune response in hiHeps upon IFN-α treatment. These results indicated that hiHeps could serve as a valuable model for anti-HBV drug screening and a potential platform for liver disease modeling.

Finally, large amounts of functional hiHeps could be generated from hHPLCs to fulfill the cell quantity demand for large-scale hepatocyte applications. The population doubling time was similar between P10 and P30 after continuously passaging hHPLCs at a 1:5 ratio (FIG. 1H), and the global gene expression profiles of hHPLCs of every 10 passages up to P40 were clustered together with hFLCs, suggesting transcriptomic stability during in vitro expansion (FIG. 1G, data not shown). These results indicated that hHPLCs could be stably expanded 9×10²⁷ times in 40 passages. Remarkably, functional hiHeps were stably generated from both early and late passages of hHPLCs and showed similar gene expression profiles and hepatocyte functions (data not shown).

Additionally, cryopreserved hHPLCs also showed stable gene expression and differentiation capabilities (FIG. 6A).

In summary, a new two-step lineage reprogramming strategy is described, that can generate large amounts of functionally competent human hepatocytes that are highly applicable for in vitro toxicity test, drug discovery and being the host for HBV. The methodological advance underlies our two-step strategy is the introduced intermediate step of first generating the proliferating plastic progenitor cells for advanced functional induction, which mimics a natural cell-fate changing route. Interestingly, the data shows that approximately 50% hepatic genes in the H3K9me3 heterochromatin region of fibroblasts, which were silent and difficult to activate by conventional reprogramming strategies, were robustly expressed in our hiHeps (FIG. 6B). This shows that our strategy could efficiently remove the epigenetic barriers to the gene regulatory network of the target cell types. Another advantage of this two-step strategy is that it allows generation of large amounts of competent cells for basic and clinical applications. The cell-fate conversion strategies disclosed herein can be applied to the cell-fate conversion of multiple cell types that can have impact on in vitro applications and regenerative medicine.

1. A method for inducing non-hepatocyte cells into hepatocytes-like cells (iHeps), comprising the steps of:
(a) treating the cell to non-hepatocyte to upregulate Hematopoietically-expressed homeobox protein (HHEX), Hepatocyte nuclear factor 4-alpha (HNF4A), Hepatocyte nuclear factor 6-alpha A (HNF6A), GATA4 and forkhead box protein A2 (FOXA2), MYC, and downregulate p53 gene expression and/or protein activity;
(b) culturing the non-hepatocyte cell in somatic cell medium
(c) expanding the cell in a hepatocyte expansion medium (HEM) comprising at least one glycogen synthase kinase (GSK) inhibitor and at least one TGFβ receptor inhibitor and
(d) culturing the cell in a hepatocyte differentiation medium comprising at least one cyclic AMP (cAMP) agonist and at least one TGFβ receptor inhibitor (2C medium).

2. The method of claim 1, comprising transfecting the cell with a vector expressing p53 SiRNA.

3. The method of claim 1, wherein the cell is cultured in somatic cell culture medium for a period of at least 7 days.

4. The method of claim 1, wherein the cell is cultured in HEM for a period of about 15 to 30 days, preferably, 20-30 days, more preferably about 20-25 days.

5. The method of claim 1, wherein the cell is cultured in hepatocyte differentiation medium for a period of at least 5 days.

6. The method of claim 1, wherein the non-hepatocyte cell is selected from the group consisting of embryonic stem cells (ESC), induced pluripotent stem cells (iPSC), fibroblast cells, adipose-derived stem cells (ADSC), neural derived stem cells, blood cells, keratinocytes and intestinal epithelial cells.

7. The method of claim 1, wherein the non-hepatocyte cell is from a mammal or the cell is optionally, a fibroblast cell.

8. The method of claim 7 wherein the mammal is selected from the group consisting of a human, rat, mouse, monkey, dog, cat, cattle, rabbit, horse, pigs.

9. (canceled)

10. The method of claim 1, wherein the TGFβ receptor inhibitor is SB431542 (4-[4-(1,3-benzodioxol-5-yl)-5-(2-pyridinyl)-1H-imidazol-2-yl]benzamide); E-616452 ([2-(3-(6-Methylpyridin-2-yl)-1H-pyrazol-4-yl)-1,5-naphthyridine].

11. The method of claim 1, wherein the cAMP agonist is forskolin or dbcAMP, or wherein the GSK inhibitor is CHIR99021 ([6-[[2-[[4-(2,4-Dichlorophenyl)-5-(5-methyl-1H-imidazol-2-yl)-2-pyrimidinyl]amino]ethyl]amino]-3-pyridinecarbonitrile])

12. (canceled)

13. The method of claim 1, wherein following culture in 2C medium, the cells are ALB+ wherein the ALB+ cells constitute over 90% of the cell population, as measured by measured by FACS analysis.

14. The method of claim 1, further comprising identifying iHeps using at least one characteristic selected from the group consisting of: (a) typical hepatocyte-morphology similar to cultured primary hepatocytes from the organism from which the non-hepatocyte cell was obtained; (b) expression of E-cadherin, Albumin (ALB) and/or hepatic transcription factors selected from the group consisting of HNF4A, HNF1A, CEBPA and CEBPB; (c) expression of key drug-metabolizing enzymes CYP450s, UGT1A1 and POR; and (d) competence for low-density lipoprotein (LDL) uptake, fatty droplets synthesis and glycogen synthesis.

15. (canceled)

16. The method of claim 1, wherein the iHep has at least one characteristic selected from the group consisting of: (a) typical hepatocyte-morphology similar to cultured primary hepatocytes from the organism from which the non-hepatocyte cell was obtained; (b) expression of E-cadherin and/or hepatic transcription factors selected from the group consisting of HNF4A, HNF1A, and CEBPA; (c) ALB+ wherein the ALB+ cells constitute over 90% of the cell population, as measured by measured by FACS analysis, for example; (d) upregulated expression of mature hepatocyte-functional genes when compared with hHPLCs and at levels comparable to expression levels in F-PHHs and/or adult liver tissue; (e) ability to maintain stably maintain expression of functional genes ALB and CYP450s genes for at least 35 days, during which fetal marker AFP, DLK1 and EPCAM expression is abolished or reduced; (f) expression of key drug-metabolizing enzymes CYP450s, UGT1A1 and POR; and (g) competence for low-density lipoprotein (LDL) uptake, fatty droplets synthesis and glycogen synthesis.

17. The method of claim 16, wherein the iHeps expresses one drug metabolizing enzyme selected from the group consisting of least one of CYP3A4, CYP2B6, CYP2C8, CYP2C9, CYP2C19, CYP2D6, CYP1A2, CYP2A6, UGT1A1 or POR, at levels comparable to the activity of the same enzyme in F-PHHs obtained from the same organism, or combinations thereof.

18. The method of claim 16, wherein MYC expression levels are lower than the levels found in hepatocytes obtained from the corresponding organism.

19. The method of claim 16, wherein the non-hepatocyte cell is a fibroblast cell, and the iHep expresses E-cadherin, and does not express the fibroblast marker genes such as COL1A1 or THY1.

20. The method of claim 16, wherein the mature functional hepatocyte gene is selected from the group consisting of ALB, AAT, CYP3A4, CYP2C9, CYP2C19, CYP2D6, CYP2A6, CYP2C8, CYP2B6, UGT1A1, UGT1A8, UGT1A10, UGT2B7, UGT2B15, NTCP, MRP2, OAT2, HNF1A, PPARA, CEBPA, CAR, RXRA, PXR, FXR, HNF4A, HNF6A, FOXA1, FOXA2, and FOXA3.

21. A bioartificial liver comprising iHeps, wherein the iHeps express a hepatocyte marker selected from the group consisting of albumin, Cytochrome P450 (CYP)3A4, CYP2B6, CYP1A2, CYP2C9, CYP2C19, or combinations thereof.

22. A kit for reprograming a non-hepatocyte cell into an iHep comprising factors for upregulating HHEX, HNF4A, HNF6A, GATA4, FOXA2 and MYC gene and factors for downregulating p53.

23. The kit of claim 22, comprising lentiviruses comprising HHEX, HNF4A, HNF6A, GATA4, FOXA2 and MYC and an oligonucleotide encoding p53 siRNA.
